(19) United States
(12) Patent Application Publication
(10) Pub. No.: US 2020/0353292 A1
(43) Pub. Date: Nov. 12, 2020

(54) AUTOMATICALLY STEERING AND FOCUSING THERAPEUTIC ULTRASOUND SYSTEMS

(71) Applicant: X Development LLC, Mountain View, CA (US)

(72) Inventors: Thomas Peter Hunt, Oakland, CA (US); Sarah LaBree Russell, Menlo Park, CA (US); Matthew Dixon Eisaman, Port Jefferson, NY (US); Benoit Schillings, Los Altos Hills (US); William Raymond Regan, San Carlos, CA (US); Alexander Zoellner, Los Gatos, CA (US)

(21) Appl. No.: 16/870,246

(22) Filed: May 8, 2020

Related U.S. Application Data
(60) Provisional application No. 62/844,866, filed on May 8, 2019.

(51) Int. Cl. A61N 7/00

(52) U.S. Cl. CPC A61N 7/00; A61N 2007/0026; A61N 2007/0078; A61N 2007/0086; A61N 2007/0052

(57) ABSTRACT

A system for therapeutic ultrasound includes one or more ultrasound transducer modules that are configured to generate ultrasound waves at a first power level and at a second power level, the first and second power levels being different and the second power level being sufficient to provide a therapeutic effect for a living subject, and an electronic controller in communication with the one or more ultrasound transducer modules, the electronic controller being programmed to cause the one or more ultrasound transducer modules to generate a first ultrasound wave at the first power level and detect a reflected ultrasound wave from the subject in response to the first ultrasound wave, determine a location of a target within the subject based on the detected reflected ultrasound wave, and, after determining the location of the target, generate a second ultrasound wave at the second power level focused at the target.

### CROSS REFERENCE TO RELATED APPLICATION

This application claims the benefit of U.S. Provisional Application No. 62/844,866, filed May 8, 2019, the contents of which are incorporated by reference herein.

### FIELD

This specification relates to therapeutic ultrasound.

### BACKGROUND

Ultrasound, or sound waves with frequencies higher than the upper audible limit of human hearing, is used widely in the medical field for imaging and therapeutic purposes. The quality of ultrasound imaging and therapy varies widely and is operator-dependent due to multiple factors, including an operator's choice of transducer, operating settings, positioning of the probe, pressure applied to the probe, amount of sonographic gel, etc. Patients may receive different standards of care from one healthcare provider and a different standard of care from another provider.

### SUMMARY

Diagnostic ultrasound, or ultrasound imaging, is a non-invasive diagnostic technique used to image inside of a patient's body. Therapeutic ultrasound, or ultrasound therapy, does not produce images, and is instead used to interact with tissues within a patient's body. Therapeutic ultrasound addresses pain conditions and promotes tissue healing through stimulation by ultrasound waves. There are two general types of ultrasound therapy available—thermal and mechanical therapy. Thermal ultrasound therapy continuously transmits ultrasound waves to cause vibrations that increase heat and metabolism in targeted tissue areas to encourage healing. Mechanical ultrasound therapy uses pulses of ultrasound waves to penetrate targeted tissue areas and cause expansion and contraction in gas bubbles within the tissue, decreasing the inflammatory response and reducing tissue swelling.

Many ultrasound systems for treating patients are susceptible to operator error. Generally, the performance of these systems is angle, pressure, and/or setting dependent, and it is difficult to ensure uniformity between imaging and treatments provided by different healthcare providers. Ultrasound therapy often requires multiple sessions throughout the course of a treatment, or in some situations, continuous treatment, and relying on the same skilled operator to administer the treatment may be too difficult or expensive to implement. Additionally, these systems typically use ultrasound at low power levels that may not provide therapeutic effects other than generating heat.

Pain treatment is an increasingly important field of technology. In some examples, ultrasound can be applied to modulate the nervous system and treat pain. A new approach to applying ultrasound to patients includes targeting a section of a patient's body and focusing the ultrasound on the targeted section to provide a therapeutic effect to the patient. The proposed method uses a first ultrasound mode for imaging purposes and using the image to direct and focus a second ultrasound mode to a target (e.g., a nerve, an organ, a muscle, a tumor, etc.). In some examples, the first (i.e., imaging) ultrasound mode corresponds to a lower power level, and the second (i.e., focused, therapeutic) ultrasound mode corresponds to a higher power level.

The focused ultrasound can be steered to the target based on the information provided by the imaging ultrasound. For example, each time a patient with an impinged nerve needs treatment, the focused ultrasound can easily be steered to the same nerve using the information provided by the imaging ultrasound. In addition to the location of the focused ultrasound, the system can adjust the phase and/or power level of the focused ultrasound based on the information provided by the imaging ultrasound. The information provided by the imaging ultrasound does not have to be comprehensible by a human, and can be provided directly to a system that interprets this information to steer and/or focus the focused, therapeutic ultrasound. The proposed method reduces opportunities for human error to affect the treatment of a patient via ultrasound, and provides a more consistent level of treatment than can be achieved by a human healthcare provider alone.

In some implementations, to effectively utilize ultrasound for medical treatment while ensuring consistent and repeatable care, a method for steering and focusing ultrasound therapy uses sensor and patient feedback to determine the appropriate intensity and location for the ultrasound waves. The proposed method can be fully automated or can be implemented as an aid to healthcare providers by providing feedback to an ultrasound operator.

The proposed method can be implemented as a therapeutic ultrasound system that includes one or more ultrasound transducer modules that are configured to generate ultrasound waves at a first power level and at a second power level, the first and second power levels being different and the second power level being sufficient to provide a therapeutic effect for a living subject, and an electronic controller in communication with the one or more ultrasound transducer modules, the electronic controller being programmed to cause the one or more ultrasound transducer modules to generate a first ultrasound wave at the first power level and detect a reflected ultrasound wave from the subject in response to the first ultrasound wave, determine a location of a target within the subject based on the detected reflected ultrasound wave, and, after determining the location of the target, generate a second ultrasound wave at the second power level focused at the target.

In some implementations, the one or more ultrasound transducer modules are housed in at least one of a wrist brace, a knee brace, a shoulder brace, a back brace, an ankle brace, a helmet, and a headphone.

In some implementations, generating the second ultrasound wave at the second power level focused at the target includes determining, based on the location of the target, at least one steering parameter for the one or more ultrasound transducer modules and dynamically adjusting, based on the at least one steering parameter, a direction of the one or more ultrasound transducer modules. Generating the second ultrasound wave at the second power level focused at the target can further include determining, based on the location of the target, the second power level and dynamically adjusting, based on the second power level, a power of the one or more ultrasound transducer modules.

In some implementations, generating the second ultrasound wave at the second power level focused at the target includes determining, based on the location of the target and a machine learning model, at least one steering parameter for the one or more ultrasound transducer modules.

In some implementations, determining a location of a target within the subject based on the detected reflected ultrasound wave includes detecting, based on the detected reflected ultrasound wave and a machine learning model, the target within the subject.

In some implementations, the one or more ultrasound transducer modules includes an imaging array that generate ultrasound waves at the first power level, the first power level being sufficient to produce an imaging effect of the target within the subject.

In another implementation, the proposed method can include directing, from one or more ultrasound transducer modules, a first ultrasound wave at a first power level to a subject, detecting a reflected ultrasound wave from the subject in response to the first ultrasound wave, determining information about a location of a target within the subject based on the detected reflected ultrasound wave, and, directing, using the one or more ultrasound transducer modules, a second ultrasound wave at a second power level focused at the target after determining the location of the target, where the second power level is sufficient to provide a therapeutic effect for the subject.

In another implementation, the proposed method can include positioning a member of an ultrasound system with respect to a living subject so that a surface of the member conforms to a body part of the living subject, receiving, at an electronic controller, information about a relative position of the member with respect to the living subject, determining, with the electronic controller, a location of a target within the living subject relative to the member, and delivering, with the ultrasound system, an ultrasound wave through the surface to the target within the living subject, the ultrasound wave having a power level sufficient to provide a therapeutic effect for the living subject.

The proposed method can be implemented as a device that conforms to the body of a patient, such as a wearable device. The device can provide the therapeutic effects of the proposed method in an easy-to-use form factor that allows patients to repeatably use the method in situations other than directly under the supervision of a healthcare provider. Additionally, the device can provide the therapeutic effects of the proposed method for longer periods than are possible when the proposed method must be performed in the presence of a healthcare provider. For example, the proposed method can be implemented in a “wearable” form—one that can be incorporated into clothing, healthcare facilities, or worn on the body. A wearable device that implements the proposed method can be worn while a patient is lying down (i.e., when the patient is asleep) for longer periods of exposure. In some instances, longer periods of exposure to the focused, therapeutic ultrasound can provide therapeutic effects different from those that can be provided in shorter periods of exposure, including those incidents in which a low-intensity but long-acting effect is desired.

Systems for implementing the proposed method can be embodied in various form factors. Devices can be customized for a particular individual or adjustable to fit a variety of patients. The proposed systems can be implemented such that patients can administer treatments without the direct assistance of a healthcare provider, allowing more frequent and longer exposures to the ultrasound therapy.

Other use cases for the proposed method include treatment by stimulation. For example, the method can be used to encourage regrowth of tissue, relaxation of tight muscles, stimulation of selected organs or glands, etc. The method can also be used to treat conditions by destroying a target. For example, the method can be used to treat a patient with tinnitus by focusing an ultrasound having sufficient power to destroy the muscle or patients having a tumor by destroying the tumor. Additional therapeutic uses are contemplated.

In one general aspect, a method for providing automatically targeted and focused therapeutic ultrasound includes directing, from an ultrasound transducer module, a first ultrasound wave at a first power level to a subject. The method includes detecting a reflected ultrasound wave from the subject in response to the first ultrasound wave. The method includes determining information about a location of a target within the subject based on the detected reflected ultrasound wave. The method also includes directing, using the ultrasound transducer module, a second ultrasound wave at a second power level focused at the target after determining the location of the target. The second power level is sufficient to provide a therapeutic effect for the subject.

In some implementations, a therapeutic ultrasound system includes an ultrasound transducer module configured to generate ultrasound waves at a first power level and at a second power level, the second power level being sufficient to provide a therapeutic effect for a living subject. The system includes an electronic controller in communication with the ultrasound transducer module, the electronic controller being programmed to cause the ultrasound transducer module to generate a first ultrasound wave at the first power level and detect a reflected ultrasound wave from the subject in response to the first ultrasound wave, determine information about a location of a target within the subject based on the detected reflected ultrasound wave, and, after determining the location of the target, and generate a second ultrasound wave at the second power level focused at the target.

In another general aspect, an ultrasound system includes a member having a surface that, during use of the ultrasound system, conforms to a body part of a living subject. The system includes an ultrasound transducer module configured to deliver, during use of the ultrasound system, an ultrasound wave through the surface to a target within the subject, the ultrasound wave having a power level sufficient to provide a therapeutic effect for the living subject. The system also includes an electronic controller in communication with the ultrasound transducer module, the electronic controller being programmed to receive information about a relative arrangement of the member with respect to the living subject and to control the delivery of the ultrasound wave to the target.

In some implementations, a method for providing automatically targeted and focused therapeutic ultrasound includes positioning a member of an ultrasound system with respect to a living subject so that a surface of the member conforms to a body part of the living subject. The method includes receiving, at an electronic controller, information about a relative position of the member with respect to the living subject. The method includes determining, with the electronic controller, a location of a target within the living subject relative to the member. The method also includes delivering, with the ultrasound system, an ultrasound wave through the surface to the target within the living subject, the ultrasound wave having a power level sufficient to provide a therapeutic effect for the living subject.

The details of one or more implementations are set forth in the accompanying drawings and the description, below. Other potential features and advantages of the disclosure will be apparent from the description and drawings, and from the claims.

The therapeutic ultrasound system provides an effective, alternative method of pain treatment without the use of narcotics or other controlled substances. The system can be used, in some implementations, by patients on an as-needed basis without supervision of a professional healthcare provider. The portable form factors in which the therapeutic ultrasound system can be implemented allow for flexibility and a wide range of applications in which patients can get pain relief.

### BRIEF DESCRIPTION OF THE DRAWINGS

FIGS. 1 and 2 are diagrams of example configurations of a steering and focusing system for therapeutic ultrasound.

FIGS. 3A and 3B are diagrams of an example focusing process for therapeutic ultrasound.

FIGS. 4A, 4B, and 4C are diagrams of example form factors of the steering and focusing system for therapeutic ultrasound.

FIG. 5 is a flow chart of an example process of the steering and focusing of a therapeutic ultrasound system.

FIG. 6 is a flow chart of an example process of administering therapeutic ultrasound using an automated steering and focusing system.

FIGS. 7A, 7B, 7C, 7D, 7E, 7F, 7G, and 7H are diagrams of example form factors of the steering and focusing system for therapeutic ultrasound.

FIG. 8 is a diagram of an example targeting process for therapeutic ultrasound.

FIG. 9 is a diagram of an example machine learning process for the steering and focusing of a therapeutic ultrasound system.

Like reference numbers and designations in the various drawings indicate like elements. The components shown here, their connections and relationships, and their functions, are meant to be examples only, and are not meant to limit the implementations described and/or claimed in this document.

### DETAILED DESCRIPTION

In general, conventional therapeutic ultrasound technology is not automatically self-tuned for particular patients and their needs, and require a skilled operator to effectively administer therapeutic ultrasound technology to a particular patient. Conventional therapeutic ultrasound technology relies on an operator to perform fine adjustments to achieve optimal operation. Typically, ultrasound systems that are used to treat patients do not automatically focus on particular targets, and rely entirely on the healthcare provider to steer and focus the ultrasound device. These treatments typically only generate heat over an operator-dependent area, and do not automatically target particular areas or structures or provide enough power to stimulate or destroy these particular areas or structures without operator input.

Among other therapeutic uses, ultrasound can be used to manage pain. The proposed techniques and systems address the problem of pain management by offering alternatives to drug treatments such as steroids, opioids, or other oral agents that are often over-prescribed and can have addictive properties. Manipulating nerve signals with ultrasound can provide patients with respite from pain, such as back pain, sciatic pain, phantom limb pain, neuropathic pain, among other types of pain. Some types of pain or discomfort can be mitigated or blocked entirely if appropriate nerves are exposed to ultrasound of a threshold intensity.

These proposed techniques and systems can be applied to treat other conditions, in addition or as an alternative to managing sources of pain. For example, therapeutic ultrasound may be used as an alternative to surgery in treating conditions such as fibroids and endometriosis, among other conditions. Therapeutic ultrasound can be used for nerve activation to disable tremors and modify bowel function; for spleen stimulation to modulate the immune system in patients with conditions such as arthritis or other autoimmune diseases; for lymph node or bone marrow stimulation; among other purposes.

Additionally, therapeutic ultrasound can be used in post-operative recovery, for example, by softening muscles, relieving pain, and enhancing bowel movements. Therapeutic ultrasound can also be used to monitor wound oxygenation and muscle tension, among other conditions. The proposed techniques and systems can stimulate targets as well as preventing propagation of signals, such as pain signals or inflammatory signals, along targets such as nerves or tissue. Stimulation of targets can range from gentle stimulation to relax muscles or enhance bowel movements to higher levels of stimulation, including destroying the target. For example, therapeutic ultrasound can be used to destroy targets such as kidney stones, nerves, blockages, or tumors, among other things. In some implementations, therapeutic ultrasound can be used to stimulate the nervous system, including peripheral nerves such as the vagus nerve, the sciatic nerve, the median nerve, and other nerves. In some implementations, therapeutic ultrasound can be applied to the brain to alter mood and focus.

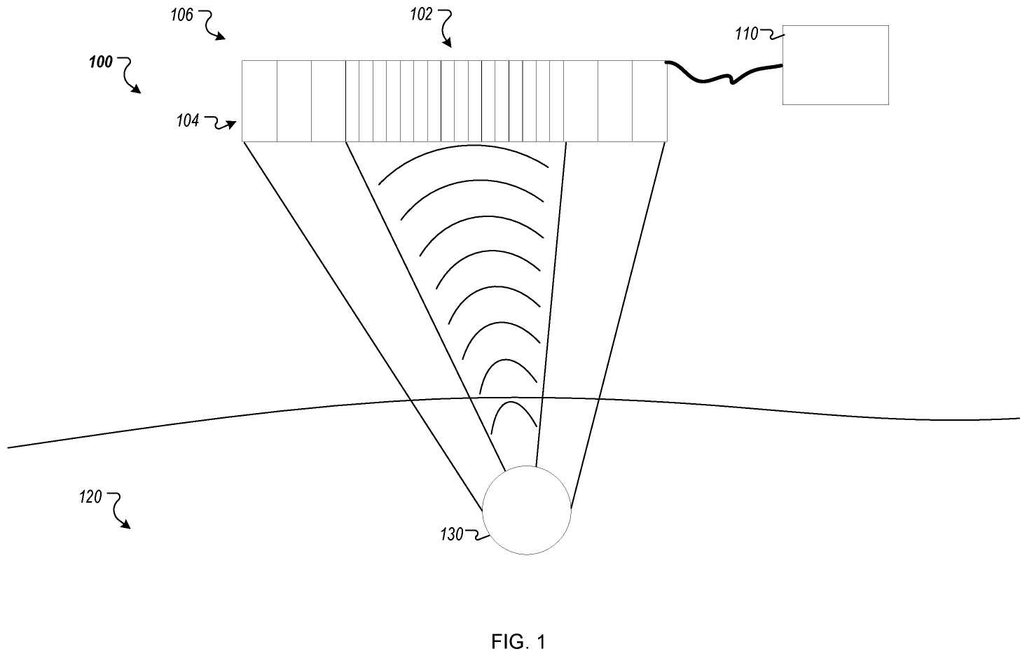

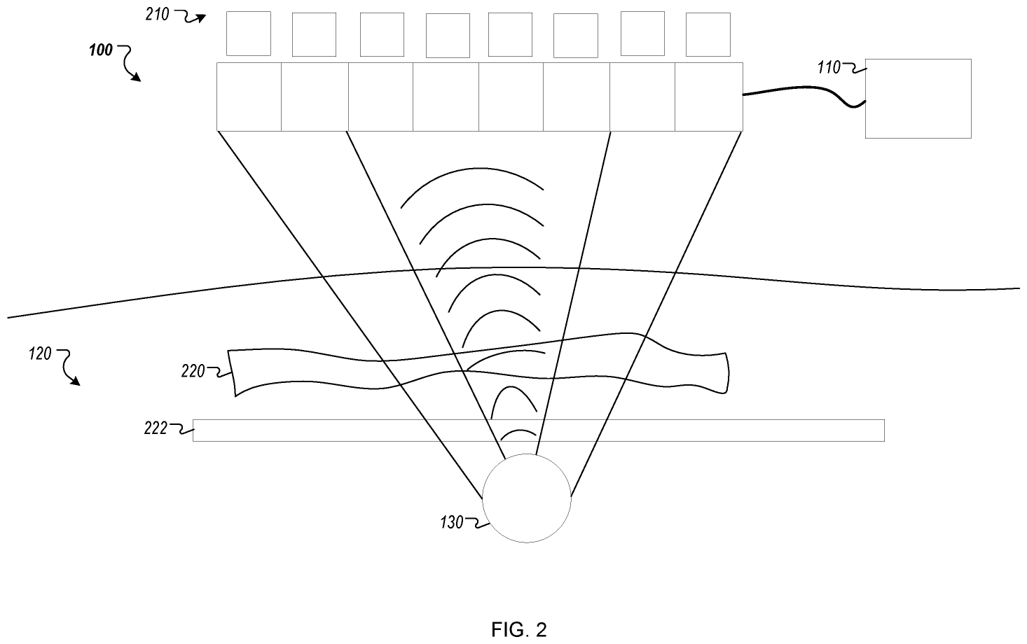

FIGS. 1 and 2 are diagrams of example configurations of a steering and focusing system for ultrasound. In these examples, systems 100 and 200 are systems that operate automatically to steer and focus ultrasound beams for therapeutic purposes. Systems 100 and 200 are configured to steer and focus an output of an array of transducers on a target. In these particular examples, systems 100 and 200 are configured to steer and focus an ultrasound output of an array of transducers on a therapeutic target inside of a patient. Systems 100 and 200 distribute pressure at a known location, or a target. Systems 100 and 200 generate an optimal pressure distribution and steer an ultrasound beam to a target inside of a patient.

System 100 is a system for automatically steering and focusing ultrasound for therapeutic purposes. System 100 includes an imaging array 102, therapeutic array 104, controller 110, patient 120, and target 130.

Imaging array 102 includes an array of transducers that produce waves that reflect from regions of differing density in a person's body, such as interfaces between different types of body tissues, to image an area of interest. Imaging array 102 can include various numbers of transducers, from a single transducer to hundreds of transducers. In this particular example, imaging array 102 includes sixteen ultrasound transducers. Imaging array 102 can include transducers that are all of one type. In some implementations, imaging array 102 can include a variety of different types of transducers that are used for imaging.

Imaging array 102 operates and collects imaging data using imaging optics mechanisms. For example, imaging array 102 can use ultrasound waves that bounce off of target 130 to image and collect data on target 130.

Therapeutic array 104 includes an array of transducers that produce waves that propagate into body tissues to treat an area of interest. Therapeutic array 104 can include various numbers of transducers, from a single transducer to hundreds of transducers. Therapeutic array 104 includes ultrasound transducers having a power level that is above a particular, predefined threshold level at which the ultrasound waves can be used only for imaging purposes. For example, imaging ultrasound frequencies can range from 1 to 20 MHz. Power levels for imaging ultrasound beams can also be calculated and tailored to a particular patient 120. Imaging ultrasound power levels can be limited in the amount of heat or effects on surrounding tissue. For example, imaging ultrasound power levels can be limited to producing less than or equal to 1° C. elevation of tissue temperature and less than or equal to 100 mW/cm² with an unfocused ultrasound beam and less than or equal to 1 W/cm² with a focused ultrasound beam. Therapeutic array 104 can include transducers that are all of one type. In this particular example, therapeutic array 104 includes six ultrasound transducers. In some implementations, therapeutic array 104 can include a variety of different types of transducers that are used for therapeutic purposes.

Imaging array 102 and therapeutic array 104 can be referred to collectively as arrays 106. In this particular example, arrays 106 are one-dimensional. The transducers within arrays 106 are not limited to performing a single function. In some implementations, transducers within arrays 106 can perform both imaging functions and therapeutic functions. Imaging array 102 can include various types of transducers, such as ultrasound transducers, electrical or radio frequency (RF) transducers, and/or x-ray transducers, among other types of transducers. For example, a single transducer within array 106 can sense an EEG reading and output therapeutic-level ultrasound. In some implementations, each transducer within arrays 106 performs a single function.

In some implementations, imaging array 102 and therapeutic array 104 can be implemented within the same array. For example, transducers capable of producing both imaging-level and therapeutic-level ultrasound can be arranged to perform both the imaging and therapeutic functions. Imaging array 102 and therapeutic array 104 can be implemented as separate arrays, where imaging array 102 includes only transducers capable of producing imaging-level ultrasound, and therapeutic array 104 includes only transducers capable of producing therapeutic-level ultrasound.

Controller 110 includes one or more computer processors that control arrays 106 to steer ultrasound output to a target, such as target 130, and control the power level of the ultrasound output. Controlling the steering of arrays 106 can include changing or maintaining a position or contact with target 130. Controlling the steering of arrays 106 can include providing a specified change in position or change in sensor values. In some implementations, controlling the steering of arrays 106 can include controlling arrays 106 to provide a specified sensor value. Controlling the power level of the ultrasound output can include changing or maintaining a power level or reflected power level from target 130. Controlling the power level of the ultrasound output of arrays 106 can include providing a specified change in power level, phase, or change in sensor values. In some implementations, controlling the power level of the ultrasound output of arrays 106 can include controlling arrays 106 to provide a specified sensor value in response to the ultrasound output.

Controller 110 is communicatively connected to arrays 106. In some implementations, controller 110 is connected to arrays 106 through communications buses with sealed conduits that meet medical hygienic standards. In some implementations, controller 110 transmits control signals to arrays 106 wirelessly through various wireless communications methods, such as RF, sonic transmission, electromagnetic induction, etc.

In some implementations, controller 110 can receive feedback from arrays 106. Controller 110 can use the feedback from arrays 106 to adjust subsequent control signals to arrays 106.

Controller 110 can be communicatively connected to sensors other than imaging array 102, and uses the data collected by the sensors and arrays 106 to steer arrays 106. In some implementations, controller 110 is connected to the sensors through communications buses with sealed conduits that meet medical hygienic standards. In some implementations, controller 110 receives sensor data from the sensors wirelessly through various wireless communications methods, such as RF, sonic transmission, electromagnetic induction, etc.

Controller 110 generates control signals for arrays 106 locally. The one or more computer processors determine control signals for arrays 106 without communicating with a remote processing system. For example, controller 110 can receive imaging data from imaging array 102, feedback data from arrays 106, and sensor data from the sensors, and process the data to determine control signals and generate control signals for arrays 106.

In some implementations, controller 110 can communicate with a remote server to receive new control signals for arrays 106. For example, controller 110 can transmit feedback from arrays 106 to the remote server, and the remote server can receive the feedback, process the data, and generate updated control signals for arrays 106.

Imaging array 102 and the sensors collect data and transmits the data to controller 110. Controller 110 uses the data from imaging array 102 and the sensors to determine control signals for arrays 106.

Imaging array 102 collects image data for patient 120. Imaging array 102 can include ultrasound transducers that image the body of patient 120 to identify the location of target 130. For example, imaging array 102 can output an ultrasound beam to an impinged nerve within patient 120 to image the nerve and determine the location of the nerve within patient 120.

Patient 120 is a human or animal patient being treated with ultrasound.

A focal spot, or target 130, is automatically targeted based on imaging. Imaging can be done using ultrasound data or other imaging data, such as EEGs and MRIs, among other types of data. Target 130 can be a therapeutic target. In this particular example, therapeutic target 130 is within the body of patient 120. Target 130 can be a structure within patient 120. For example, target 130 can be a muscle, a nerve, an organ, a blood vessel, an area of tissue, a mass within patient 120, a section of bone, among other objects.

System 200 is a system for automatically steering and focusing ultrasound for therapeutic purposes. System 200 includes a multidimensional transducer array 210 and non-uniform media 220 and 222.

Transducer array 210 can be an implementation of arrays 106. Transducer array 210 can be a multidimensional array of transducers. In some implementations, transducer array 210 includes imaging transducers and therapeutic transducers as described above with respect to FIG. 1. In this particular implementation, transducer array 210 is shown to be a 2×8 array of transducers. Transducer array 210 can include various numbers of transducers in various arrangements. For example, transducer array 210 can include a 23×4 array of transducers, where the first 23 transducers are all of the same type of transducer, and perform imaging functions; the second 46 transducers are of different types and perform imaging functions different from each other and from the first 12 transducers; and the last 23 transducers are of two different types and perform therapeutic functions.

Transducer array 210 can be arranged such that the imaging and therapeutic transducers are separate. For example, an existing imaging array could be placed in front of a therapeutic array such that a system for imaging can be retro-fit with a therapeutic array to form a system, such as system 100 or 200, that automatically steers a therapeutic ultrasound beam to a target and automatically focuses the therapeutic ultrasound beam on the target.

Controller 110 can control and interact with transducer array 210 as described above with respect to arrays 106 of FIG. 1. Additionally, controller 110 can be communicatively connected to transducer array 210 as described above with respect to arrays 106 of FIG. 1.

Non-uniform media 220 and 222 can be objects within patient 120 that are non-uniform and can be difficult to image. In this particular example, non-uniform media 220 is a layer of fat, and non-uniform media 222 is a nerve. Non-uniform media 220 and 222 can be various other objects or structures that do not have homogeneous wave propagation properties. For example, non-uniform media 220 and 222 can be fat, nerves, and blood vessels, among other things.

In order to image objects or structures that do not have homogeneous wave propagation properties, the proposed imaging systems utilize techniques such as phase reconstruction.

### Steering

Controller 110 automatically steers arrays 106 and/or transducer array 210 the ultrasound beam. Controller 110 uses an algorithm to automatically identify and steer arrays 106 and/or transducer array 210 to target 130 based on imaging data, including data collected by imaging array 102. For example, patient 120 can be prescribed ultrasound therapy to treat a particular nerve 130. Controller 110 can use an algorithm, such as a machine learning algorithm, to automatically identify the particular nerve 130 within patient 120 and steer an ultrasound beam produced by therapeutic array 104 and/or transducer array 210 to the automatically identified nerve 130.

Controller 110 can use variation in a pulse echo to steer the arrays 106 and/or transducer array 210. For example, controller 110 can use focused ultrasound pulse-echo may be used to modify the control signals for steering the arrays 106 and/or transducer array 210. In some implementations, controller 110 can use imaging pulses interleaved between the focused ultrasound pulses to repeatedly acquire the location of target 130. For example, controller 110 can automatically and continuously check, using imaging pulses fired between ultrasound pulses to determine the location of target 130 in situations where patient 120 moves or target 130 shifts. Patients cannot stay perfectly still at all times, and it is possible for targets within patients to shift or naturally drift even if the patient is relatively motionless.

Controller 110 can steer the output of arrays 106 and/or transducer array 210 based on feedback data for the output beam. For example, controller 110 can use the contrast of the strength of return pulses with the strength of the output beam. Controller 110 can use the strength of the return pulse from the focused ultrasound pulses to determine whether to update or alter the control signal for the output beam.

Controller 110 can use data that is not understandable by humans. For example, controller 110 can automatically apply signal processing to analyze the data and determine how to steer and focus the output of arrays 106 and/or transducer array 210. Controller 110 can optimize steering of arrays 106 and/or transducer array 210 by using, for example, a trained neural network to follow target 130. For example, controller 110 can use simulations and models, among other training methods, to train a machine learning model. Controller 110 can use other algorithms and methods to analyze the data and determine how to steer and focus the output of arrays 106 and/or transducer array 21, including image recognition and machine vision, among other techniques.

Controller 110 can use machine learning models which accept sensor data collected by arrays 106 and transducer array 210 and/or other sensors as inputs. The machine learning models may use any of a variety of models such as decision trees, linear regression models, logistic regression models, neural networks, classifiers, support vector machines, inductive logic programming, ensembles of models (e.g., using techniques such as bagging, boosting, random forests, etc.), genetic algorithms, Bayesian networks, etc., and can be trained using a variety of approaches, such as deep learning, perceptrons, association rules, inductive logic, clustering, maximum entropy classification, learning classification, etc. In some examples, the machine learning models may use supervised learning. In some examples, the machine learning models use unsupervised learning.

Ultrasound wave propagation is not affected by the strength of the ultrasound beam. Because increasing the power or focusing the beam does not change the manner in which ultrasound waves travel through a patient or reflect from the patient, the steering control signals do not need to account for the difference in power between an imaging array of transducers, such as imaging array 102 or transducer array 210, and a therapeutic array of transducers, such as therapeutic array 104 or transducer array 210. Thus, the location of target 130 determined using imaging array 102 or transducer array 210 can be used to determining steering control signals for therapeutic array 104 or transducer array 210.

Controller 110 can accept input other than imaging data to determine steering control signals for arrays 106 and/or transducer array 210. The input can include sensor data from sensors separate from systems 100 and 200, such as temperature sensors, light sensors, heart rate sensors, and blood pressure monitors, among other types of sensors. In some implementations, the input can include patient input. A patient can steer the beam based on the patient's own perception of the location of the target. For example, the patient can provide verbal feedback to a healthcare provider operating systems 100 and/or 200 to direct the therapeutic ultrasound beam to the appropriate location for pain suppression. Patients can provide input through other techniques, including input through a computer graphical interface or a tactile interface, among other techniques. In some implementations, controller 110 receives sensor information regarding the conditions of patient 120 and target 130. For example, sensors monitoring the size of a particular mass 130 within a blood vessel in patient 120 can provide input to controller 110. Controller 110 can use this sensor data to automatically steer arrays 106 and/or transducer array 210 to direct the output ultrasound beam to remaining portions of the particular mass and destroy the entire mass.

Controller 110 uses feedback data from sensors, patients, and arrays 106 and transducer array 210 to create a closed loop operation that continually and automatically controls arrays 106 and/or transducer array 210 to steer therapeutic ultrasound beams to target 130.

### Focusing

Each transducer of arrays 106 and/or transducer array 210 can be a broadband transducer. In some implementations, multiple transducers with various ranges of frequencies can be included in a single array to cover a wide frequency spectrum from around 500 kHz to around 20 MHz. For example, transducers in therapeutic array 104 can have a frequency of 1 MHz. Therapeutic ultrasound transducers of therapeutic array 104 and/or transducer array 210 have higher intensity and power than imaging and diagnostic ultrasound transducers. For example, therapeutic ultrasound transducers of therapeutic array 104 and/or transducer array 210 can have a power level up to 5 W/cm², and can be capable of producing coagulation necrosis of tissue and used for ablation. In some implementations, therapeutic ultrasound transducers of therapeutic array 104 and/or transducer array 210 can have a power level between 0.1 W/cm² and 3 W/cm² and can be capable of producing low intensity, therapeutic effects that are nondestructive. For example, therapeutic ultrasound transducers of therapeutic array 104 and/or transducer array 210 can have power levels greater than 100 mW/cm2 with an unfocused ultrasound beam and greater than 1 W/cm² with a focused ultrasound beam.

Each therapeutic ultrasound beam can have a particular “spot size” or resolution. This resolution is the size of the ultrasound beam at target 130. The therapeutic ultrasound beam resolution can range from around 10 μm/MHz to around 1 mm/MHz. For example, the resolution of a particular therapeutic ultrasound beam can be 100 μm/MHz. The resolution of a therapeutic ultrasound beam is limited by the frequency of the ultrasound beam: Higher frequencies allow for a smaller resolution, and lower frequencies provide a larger resolution.

The pressure, or power level, of a therapeutic ultrasound beam can be measured in W/m². The pressure for a therapeutic ultrasound beam can range from a reversible damage threshold, where any effects of the ultrasound beam on target 130 can be reversed, to above a threshold where damage to target 130 is irreversible. Systems 100 and 200 can use pressures that do not rise above the damage threshold of a particular target 130 when providing such treatments as stimulating target 130. For example, system 100 and 200 can use pressures that do not rise above the thermal or cavitation damage thresholds for human tissue. Systems 100 and 200 can use pressures that rise above the damage threshold of a particular target 130 when providing such treatments as destroying target 130. For example, where destroying a target 130 is desirable, systems 100 and 200 can use pressures that meet or exceed the damage threshold for a particular type of target 130, such as a kidney stone, fibroid, or a nerve among other types of targets. For example, systems 100 and 200 can use high pressure ultrasound beams to perform ablation of a nerve to stop epilepsy.

Controller 110 can calculate the appropriate phases for therapeutic ultrasound beams that have been steered to target 130. These phases can interact to increase or decrease resolution and/or power, and can be calculated automatically using various algorithms, including machine learning algorithms as described above. Controller 110 can automatically determine appropriate phases by changing phases for the therapeutic ultrasound output of arrays 106 and/or transducer array 210 and use an amount of power returned from target 130 to determine whether to change the pressure. For example, controller 110 can use the amount of power returned from a muscle 130 being stimulated by therapeutic ultrasound pulses, and automatically determine a change to the power level of the therapeutic ultrasound. Controller 110 can use, for example, phased arrays that emit ultrasound pulses and adjust the phases of these pulses for maximum intensity, up to a predetermined safety threshold level.

In some implementations, there is a holographic projection of the focal spot of the ultrasound beam that is used for beamforming. The projection of the focal spot can be a visualization of the location of target 130. Controller 110 can use a signal processing technique with sensors and arrays 106 and/or transducer array 210 for beamforming. Controller 110 achieves directional signal transmission or reception through beamforming by combining elements in an antenna array such that signals at particular angles experience constructive interference while others experience destructive interference in order to achieve spatial selectivity. Based on the ultrasound imaging or measurements, systems 100 and 200 can match propagation delays to the target from each element in the phased array. For example, the array can be one-dimensional as in system 100 or multi-dimensional as in system 200, such that the ultrasound waves arrive at the target in-phase and in-focus. The directional transmission and focus process is controlled through a technique similar to phase reconstruction for imaging techniques, but with the specific aim of maximizing delivered energy to the target through complex media without homogeneous propagation properties.

Systems 100 and 200 can provide different shapes of focal points. For example, systems 100 and 200 can provide an elongated focus that is not simply circular. Controller 110 can control arrays 106 and/or transducer array 210 to provide different shapes of focal points by, for example, steering individual transducers of arrays 106 and/or transducer array 210. Systems 100 and 200 can provide different shapes of focal points such as rectangular focal points, oblong focal points, linear focal points, and triangular focal points, among other shapes. In some implementations, systems 100 and 200 can provide focal points that are elongated to span a particular area. For example, systems 100 and/or 200 can provide an oval focal point that spans an area along a patient 120's nerve.

To maximize the effect of the ultrasound therapy on a particular target while minimizing the dosage of ultrasound power at a particular location, the therapeutic ultrasound beam used to treat the particular target can be spread out in one dimension and not the other dimension. Systems 100 and 200 can be used for to treat targets that are longer in one dimension than the other. For example, a nerve can be longer along a patient's arm and shorter across the patient's arm, and controller 110 can control arrays 106 and/or transducer array 210 such that the ultrasound beam can be spread out along a nerve fiber. This can be done with axicon—a special type of lens that has a conical surface and transforms beams into ring shaped distribution—zone plates or Soret—an intense peak in the blue wavelength region of the visible spectrum—zone plates integrated with the transducers.

In some implementations, controller 110 can control arrays 106 and/or transducer array 210 to stimulate different focal spots 130 and can select different targets. For example, controller 110 can focus on or along two different points of a particular nerve using a two-dimensional phased array 210 that can adjust the X and Y coordinates of the focal points 130.

In some implementations, systems 100 and 200 can provide multiple focal points. For example, controller 110 can control arrays 106 and/or transducer array 210 to provide one focal point per a cluster of transducers or per transducer. These ultrasound beams can be spaced apart on a scale that ranges between nm and cm. In some implementations, controller 110 controls arrays 106 and/or transducer array 210 to simultaneously stimulate two or more spot targets 130. For example, controller 110 can control phased arrays 210 to produce a beam pattern that focuses on multiple targets 130. In some implementations, systems 100 and 200 can provide multiple focal points using different transducers or arrays to target multiple separate points along a single target. For example, controller 110 can control arrays 106 and/or transducer array 210 to target multiple separate points along a single nerve for additional therapeutic benefits.

In some implementations, systems 100 and 200 can focus multiple transducers on a single target 130. For example, controller 110 can control arrays 106 and/or transducer array 210 to sync pulses from multiple transducers to match, for example, a measured speed of a pain signal influx.

Systems 100 and 200 can provide multi-pulse superposition. A pulse at a single focal point makes a pressure wave that propagates radially outward. Systems 100 and 200 can control the therapeutic ultrasound beams to stack the radially propagating pulse with a second pulse at a new position along target 130. Controller 110 can control arrays 106 and/or transducer array 210 to move the transducers to the new position. In some implementations, arrays 106 and/or transducer array 210 can have multiple focal points. Controller 110 can control the steering and focus of the superpositioned ultrasound pulses such that single-pulse thresholds for power are respected while building up displacement with pressure or shear waves from multiple pulses with different focal locations.

Systems 100 and 200 can provide confirmation of targeting and enable closed loop actuation of the therapeutic ultrasound system. Systems 100 and 200 can provide an indication of targeting through, for example, visual or tactile feedback, among other techniques. For example, systems 100 and 200 can provide electrical confirmation in the form of an electromyography (EMG) signal read out by sensors to measure the effectiveness of nerve stimulation through therapeutic ultrasound.

In some implementations, systems 100 and 200 can provide electrical stimulation and ultrasound stimulation. For example, controller 110 can control arrays 106 and transducer array 210 to effect surface electrical stimulation at a patient 120's skin and provide ultrasound beam stimulation at a target spot 130 within patient 120.

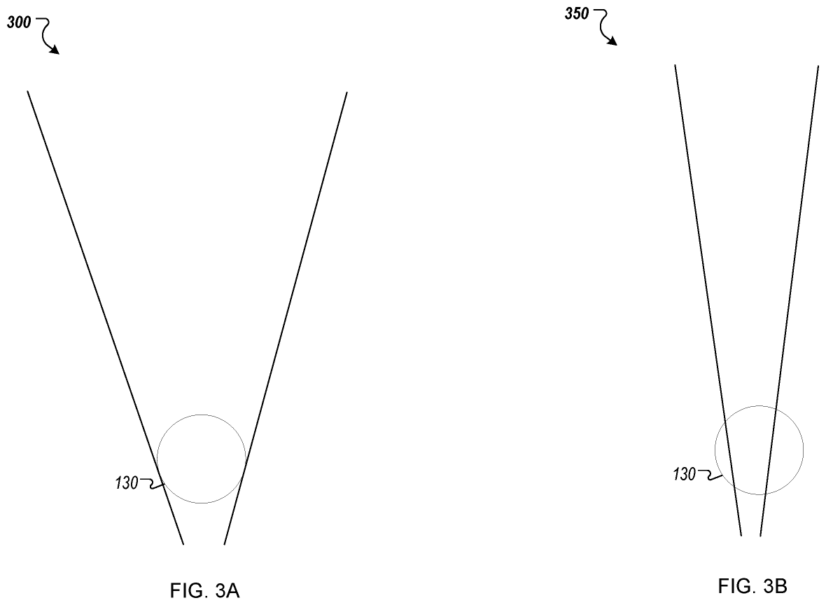

FIGS. 3A and 3B are diagrams of an example focusing process for therapeutic ultrasound systems, such as systems 100 and 200 as described in FIGS. 1-2.

FIG. 3A is a diagram of an example focusing process 300 in which the therapeutic beam (e.g., from systems 100 and/or 200) is focused past the therapeutic target such that the beam size at a target fully encompasses the target and minimizes the effects on surrounding tissue.

Controller 110 of systems 100 and 200 can provide a defocused beam. In some implementations, the therapeutic beams from systems 100 and 200 are high-frequency ultrasound beams that are purposely defocused such that the entire width of the target can be encompassed with sharp boundaries. For example, a nerve target 130 of around 500 μm can be encompassed with a defocused portion of an ultrasound beam having a spot size smaller than 500 μm.

In some implementations, systems 100 and 200 use, in addition to or instead of using high frequency ultrasound beams, a zone plate controlled by controller 110 for focusing and use mechanical or electrical actuation and/or flexing of the zone plate to control the defocusing and modulation of the beam at mechanically attainable frequencies. Zone plates can be integrated with arrays 106 and/or transducer array 210 for focusing output beams, such as ultrasound beams, and can be considered a part of arrays 106 and/or transducer array 210.

FIG. 3B is a diagram of an example focusing process 350 in which the therapeutic beam (e.g., from systems 100 and/or 200) is focused such that only a portion of a target is encompassed within the ultrasound beam. This narrowed focus can be useful, for example, to stimulate only a portion of a nerve 130 or destroying a blockage 130 within a blood vessel, among other situations.

In some implementations, systems 100 and/or 200 use, in addition to or instead of using high frequency ultrasound beams, a zone plate controlled by controller 110 for focusing and use mechanical or electrical actuation and/or flexing of the zone plate to control the narrowing and modulation of the beam at mechanically attainable frequencies.

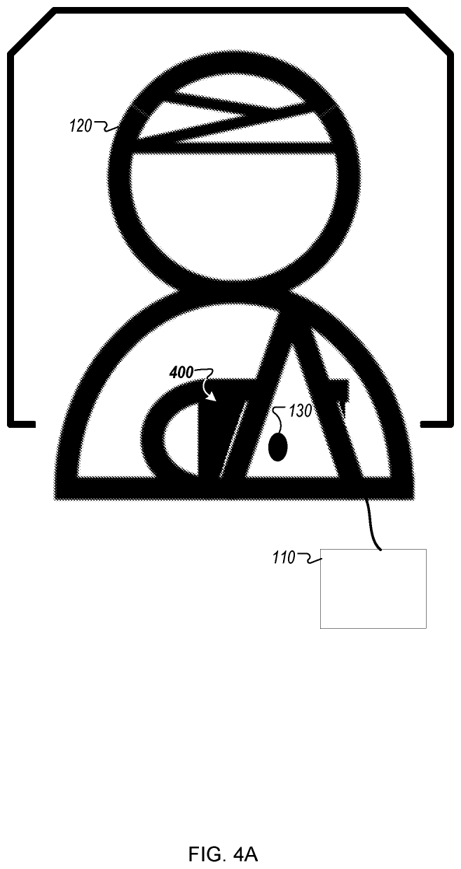

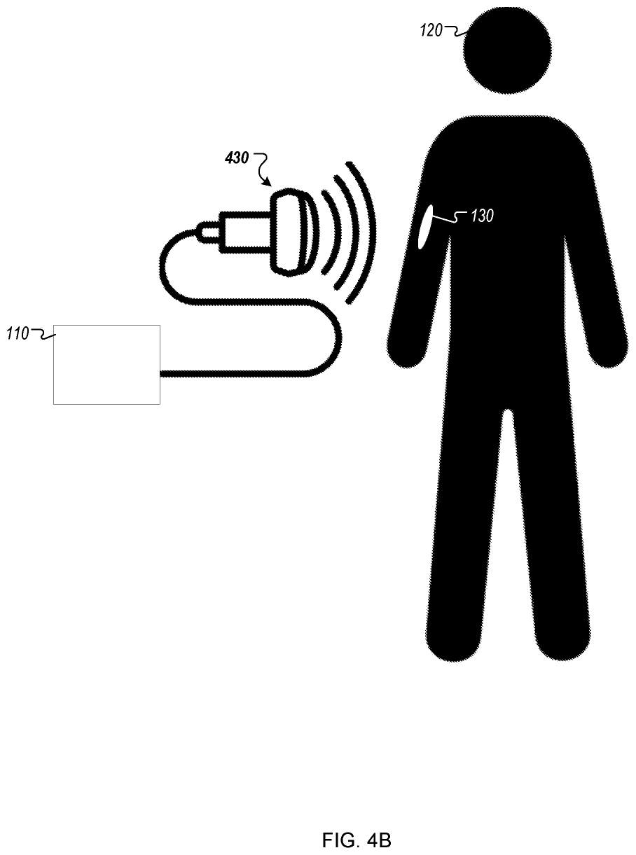

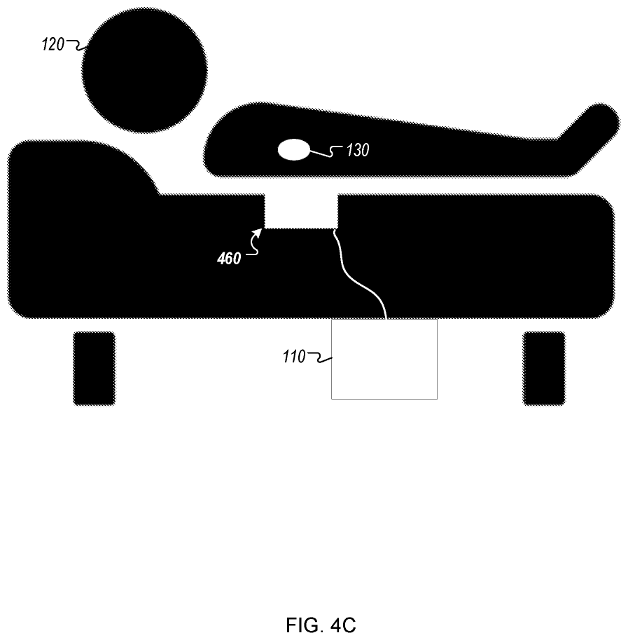

FIGS. 4A, 4B, and 4C are diagrams of example form factors of the steering and focusing systems, such as systems 100 and 200, for therapeutic ultrasound as described in FIGS. 1-3B.

FIG. 4A illustrates an example device 400 that can be worn by a patient, such as patient 120. Device 400 includes arrays 106 and/or transducer array 210, and can be an implementation of systems 100 and/or 200 as described above with respect to FIGS. 1-3B. Device 400 can include all capabilities of devices 430 and 460 in a different form factor.

Device 400 can be easily administered to patient 120, and can be worn by a mobile patient. For example, patient 120 may be able to walk around with device 400. Device 400 can be powered by a power system that is integrated with device 400. For example, device 400 can be battery powered. In some implementations, device 400 can be powered by a separate power system that can be connected with common power sources such as outlets. In some implementations, device 400 can be powered by a power system integrated with controller 110. For example, controller 110 can include an integrated power system and controller 110 can be integrated with device 400 such that patient 120 is able to move while wearing device 400.

In this particular example, device 400 is shown to be administered in a healthcare provider setting. Device 400 can also be self-administered, and may be worn outside of the context of the healthcare provider's office. When device 400 can be self-administered, device 400 allows patient 120 to continue ultrasound therapy at home and can be worn for longer periods of time. Thus, patient 120 can utilize the treatment without requiring the healthcare provider to administer the treatment.

In some implementations, if device 400 is operated without the supervision of a healthcare provider, device 400 may allow for only one position of automatically adjusting ultrasound beam position for patient safety. For example, if patient 120 operates device 400 at home, device 400 may only be worn on a patient's leg, arm, or foot, among other parts.

Device 400 can be customized for a particular individual. For example, device 400 can be cast to a specific patient's body such that the transducer is in a fixed location relative to the specific patient's body. Device 400 can be manufactured in a variety of ways, including additive manufacturing, machining, injection moulding, casting, or plastic processing, among other methods.

In some implementations, controller 110 can control transducers within device 400 using fiducials such as bones or joints, among other structures, based on imaging of the patients' bodies. For example, controller 110 can receive sensor data from various sensors, including imaging sensors, and use the imaging data of patient 120's elbow to automatically generate control signals for transducers, such as arrays 106 and/or transducer array 210 within device 400.

In some implementations, device 400 can be used in a healthcare provider setting and can be provided to multiple different patients. Controller 110 can adapt to the different bodies of different patients using imaging data of each patient. For example, a hospital can implement a lending program for ultrasound therapy systems, such as device 400, that is administered to different patients with different needs.

Device 400 can be made of flexible and/or adaptable material that wraps around an area of a patient's body. For example, device 400 can be implemented as a fabric cuff around patient 120's torso for stimulation of patient 120's abdominal muscles. Transducers within device 400 can have independent actuation from other transducers, and controller 110 can control the timing of the independently actuated transducers to steer and focus therapeutic ultrasound beams to the desired therapeutic target, such as target 130. Automatically steered and focused ultrasound systems as described in FIGS. 1-3B can be embedded in flexible fabric that can be molded to fit an area of a patient's body. For example, the fabric can be molded to fit a patient's head, arm, torso, or leg, among other areas. In some implementations, various standard models can be created and then adapted to a particular patient. For example, a portion of a cast can be created through additive manufacturing, and the cast can be completed when it is applied to patient 120.

Device 400 can be coupled to the patient's skin through various mechanisms, including fluids or gel, among other media. Any conductive substance with appropriate acoustic conducting properties and provides appropriate acoustic coupling to a patient's skin can be used. For example, device 400 can be a flexible cuff coupled to patient 120's skin through an adhesive gel. Device 400 can be a fluid-filled pillow or an inflatable cuff (inflatable with fluids such as water or gel, among other fluids) that is held to a target area 130 of patient 120. In some implementations, device 400 can be physically held against patient 120's skin by a healthcare provider or patient 120 and coupled to patient 120's skin by a fluid or gel.

In some implementations, device 400 can be implemented as a patch that doesn't require other forms of attachment. For example, device 400 can be an adhesive “hands-free” patch. In another example, device 400 can be a perforated patch with temporary glue for coupling to patient 120's skin. For example, the glue can break down after a predetermined period of time.

In some implementations, device 400 can be implemented as a flexible device with rigid portions that have compliance, and that are pushed into patient 120 with sufficient force such that patient 120's body tissue conforms to the rigid portions. For example, device 400 can be a patch made of flexible materials or fabrics to allow the device to conform and couple to patient 120's body.

Device 400 can be calibrated after it is administered to patient 120. For example, controller 110 can perform a calibration after fitting device 400 to patient 120 by using ultrasound reflections from fiducials to calibrate the steering and focusing of therapeutic ultrasound beams output from device 400. For example, if device 400 is molded to a particular patient 120, patient 120 must return to the administering healthcare provider's facilities for multiple sessions, patient 120 may not put on the device in the same way every time. Controller 110 can use reflections from patient 120's skull to account for event to event fitting differences for patient 120. Controller 110 can also perform calibrations for a same device 400 that is used for multiple different patients. For example, controller 110 can control a communal device 400 to account for variation from patient to patient in characteristics such as bone thickness and/or shape and differences in fat and muscle location and distribution, among other characteristics.

Other sensors, such as EEG and EMG sensors, among other types of sensors, can be embedded in device 400 to allow quantification of desired effect and appropriate feedback and adjustment to device 400 and its therapeutic ultrasound output.

In some embodiments, device 400 can include mechanically and/or electrically adjustable zone plates, or devices used to focus light or other things exhibiting wave characteristics using diffraction instead of refraction or reflection, controlled by controller 110, that are embedded within each transducer. Controller 110 controls these zone plates to focus the radiation to the target.

FIG. 4B illustrates example device 430 that can be administered by a healthcare provider. Device 430 includes arrays 106 and/or transducer array 210, and can be an implementation of systems 100 and/or 200 as described above with respect to FIGS. 1-3B. Device 430 can include all capabilities of devices 400 and 460 in a different form factor.

Controller 110 can automatically provide feedback regarding steering and focus inputs required from a healthcare provider operating device 430. Controller 110 can provide visual, tactile, or audible feedback, among other types of feedback while a healthcare provider is operating device 430. For example, controller 110 can vibrate in an intuitive way to indicate the automatic steering and focusing control signals for device 430. Patient 120 can also provide feedback to the operator of device 430 as described above with respect to FIGS. 1-3B and 4A, creating a closed feedback loop.

In this particular implementation, device 430 is a rigid device and has the automatic steering and focusing systems as described in FIGS. 1-3B and 4A. In some implementations, device 430 can include a rigid probe that is pushed into patient 120 with a sufficient force such that patient 120's body tissue is compliant to the surface of the probe. Controller 110 can analyze and account for imaging and/or sensor data indicating device compliance as well as compliance of patient 120's body tissue. For example, device 430 can be a wand that can be moved and placed on target areas of a patient by an operator. In some implementations, device 430 can be operated by patient 120 without the supervision of a healthcare provider.

FIG. 4C illustrates example device 460 that can be administered by the healthcare provider. Device 460 includes arrays 106 and/or transducer array 210, and can be an implementation of systems 100 and/or 200 as described above with respect to FIGS. 1-3B. Device 460 can include all capabilities of devices 400 and 430 in a different form factor.

In this particular implementation, device 460 is integrated with hospital equipment, and patient 120 can lie on device 460. For example, device 460 can be a device integrated into a position on a hospital bed on which patient 120 lies during recovery. In some implementations, device 460 can be used outside of the context of the healthcare provider's office. For example, device 460 can be integrated with a patient's bed at the patient's own home, among other situations. Device 460 allows a patient to recover without being restricted to a healthcare provider's office or facilities. Device 460 can automatically steer and focus therapeutic ultrasound beams to a target 130 within patient 120's body.

Device 460 can be used for applications such as surgery recovery and rehabilitation, among other situations. Patients 120 can use device 460 for therapeutic purposes without waiting for a healthcare provider to administer the therapeutic ultrasound beams to a target area.

In some embodiments, device 460 can be implemented as multiple small transducers with each transducer being individually controlled. For example, device 460 can be implemented in a form factor similar to a “bed of pins”, where each pin is a transducer head and is pressure sensitive, conforming to a patient's body and collecting imaging and other sensor data. In some implementations, device 460 implemented as a bed of pins has a single ultrasound source that can sweep through the entire array of “pins,” or transducer heads, similar to electron-beam scanning across a cathode ray tube. In some implementations, each “pin” of device 460 includes a piezo element.

Devices 400, 430, and 460 can be used for simultaneous ultrasound data collection and ultrasound therapy, for example, to identify and track the location of various target structures or objects 130 within a patient 120. For example, a patient can lie on device 460 implemented as a bed of pins, and device 460 can simultaneously image patient 120's kidney while device 460 outputs ultrasound beams that destroy kidney stones 130 within the kidney.

Other example form factors are described below.

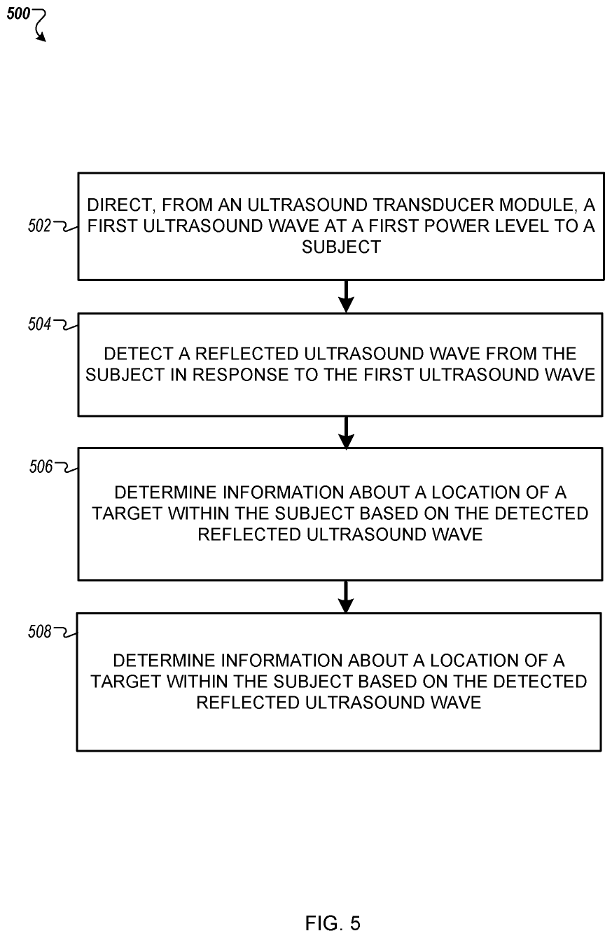

FIG. 5 is a flow chart of an example process 500 of the steering and focusing of a therapeutic ultrasound system. Process 500 can be implemented by therapeutic ultrasound systems such as systems 100 and 200 as described above with respect to FIGS. 1-3B. In this particular example, process 500 is described with respect to system 100. Process 500 can be performed using machine learning models that are described in further detail below.

Briefly, according to an example, the process 500 begins with directing, from an ultrasound transducer module, a first ultrasound wave at a first power level to a subject (502). For example, controller 110 can direct, from imaging array 102, a first ultrasound wave at a first power level, such as a diagnostic imaging level, to a target 130 within patient 120.

The process 500 continues with detecting a reflected ultrasound wave from the subject in response to the first ultrasound wave (504). For example, controller 110 can detect a reflected ultrasound wave from target 130 in response to the first ultrasound wave from imaging array 102.

The process 500 continues with determining information about a location of a target within the subject based on the detected reflected ultrasound wave (506). For example, controller 110 can determine information about a location of target 130 within patient 120 based on the detected reflected ultrasound wave from imaging array 102.

The process 500 concludes with directing, using the ultrasound transducer module, a second ultrasound wave at a second power level focused at the target after determining the location of the target, wherein the second power level is sufficient to provide a therapeutic effect for the subject (508). For example, controller 110 can direct a second ultrasound wave from therapeutic array 104 at a second power level at target 130 after determining the location of target 130. The second power level can be a power level higher than power levels used for imaging or diagnostic, and can provide a therapeutic effect for patient 120 without exceeding a power level threshold at which unintended damage occurs.

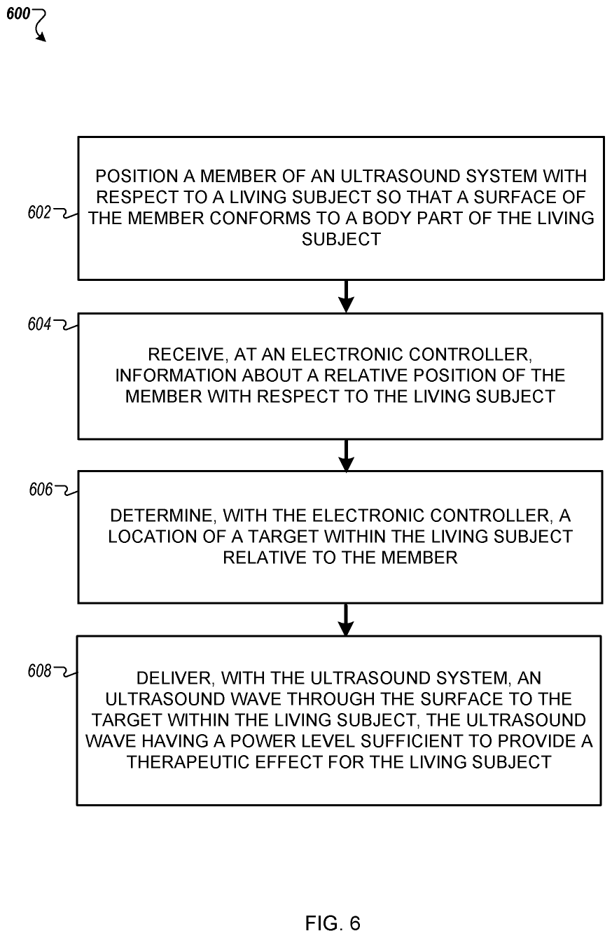

FIG. 6 is a flow chart of an example process 600 of administering therapeutic ultrasound using an automated steering and focusing system. Process 600 can be implemented by therapeutic ultrasound systems such as systems 100 and 200 as described above with respect to FIGS. 1-3B, and device form factors such as devices 400, 430, and 460 as described above with respect to FIGS. 1-4C. In this particular example, process 600 is described with respect to system 100 and device 400. Process 600 can be performed using machine learning models that are described in further detail below.

Briefly, according to an example, the process 600 begins with positioning a member of an ultrasound system with respect to a living subject so that a surface of the member conforms to a body part of the living subject (602). For example, device 400 can be positioned with respect to patient 120 such that the surface of device 400 containing arrays 106 conforms to a leg of patient 120's within which target 130 exists.

The process 600 continues with receiving, at an electronic controller, information about a relative position of the member with respect to the living subject (604). For example, controller 110 receives information about a relative position of device 400 with respect to patient 120 through data from sensors and arrays 106.

The process 600 continues with determining, with the electronic controller, a location of a target within the living subject relative to the member (606). For example, controller 110 determines a location of target 130 within patient 120 relative to a fiducial within patient 120, such as patient 120's knee.

The process 600 concludes with delivering, with the ultrasound system, an ultrasound wave through the surface to the target within the living subject, the ultrasound wave having a power level sufficient to provide a therapeutic effect for the living subject (608). For example, controller 110 generates control signals such that device 400 delivers an ultrasound wave through the surface of patient 120's body to target 130 within patient 120's leg, where the ultrasound wave is above a power level threshold at which the ultrasound wave can only be used for diagnostic or imaging purposes, but below a power threshold at which unintended damage is caused to patient 120.

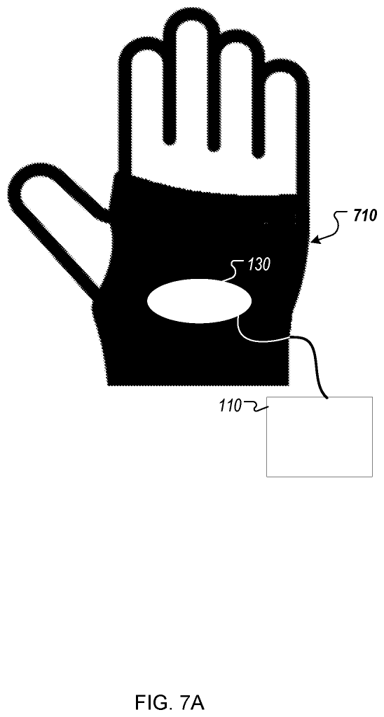

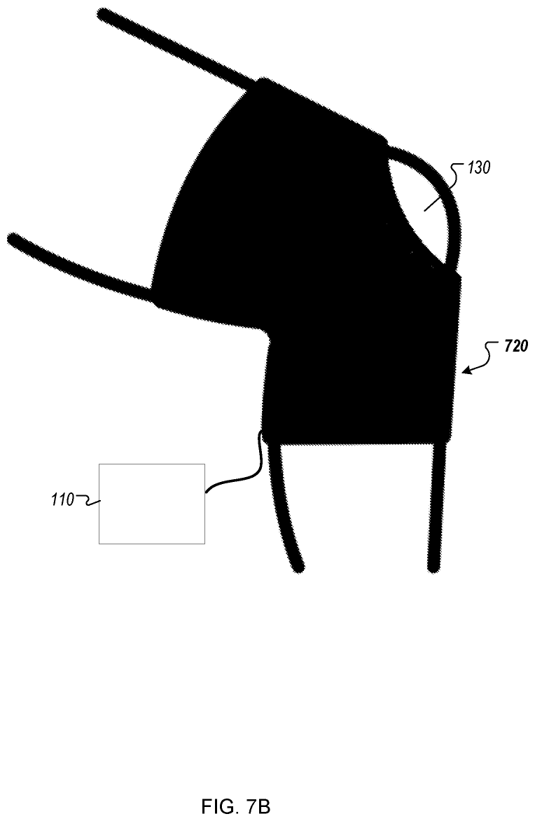

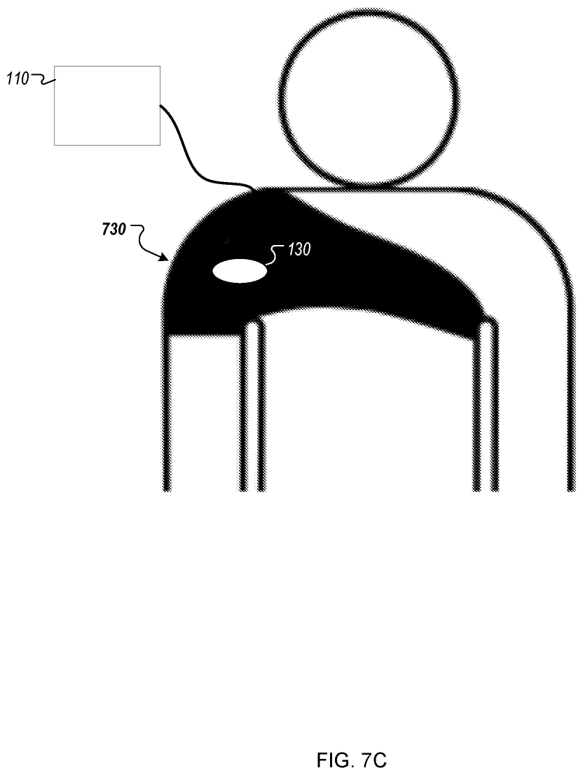

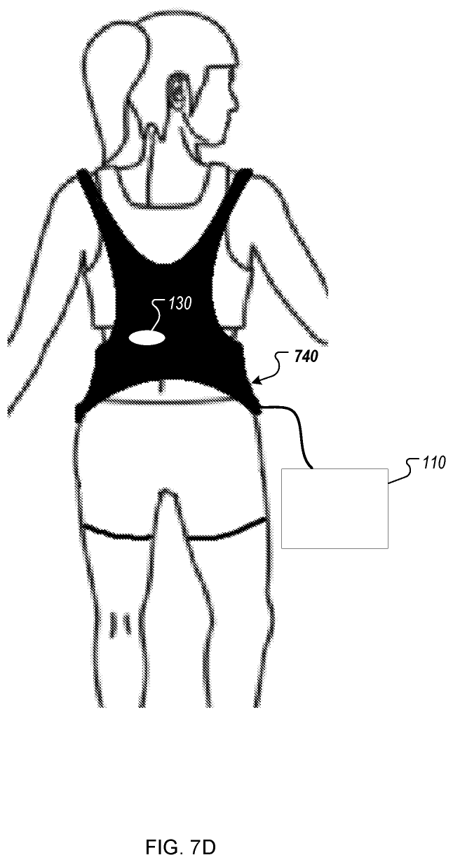

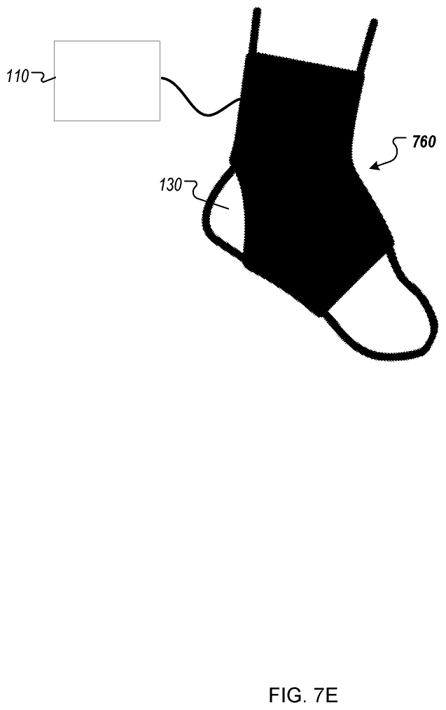

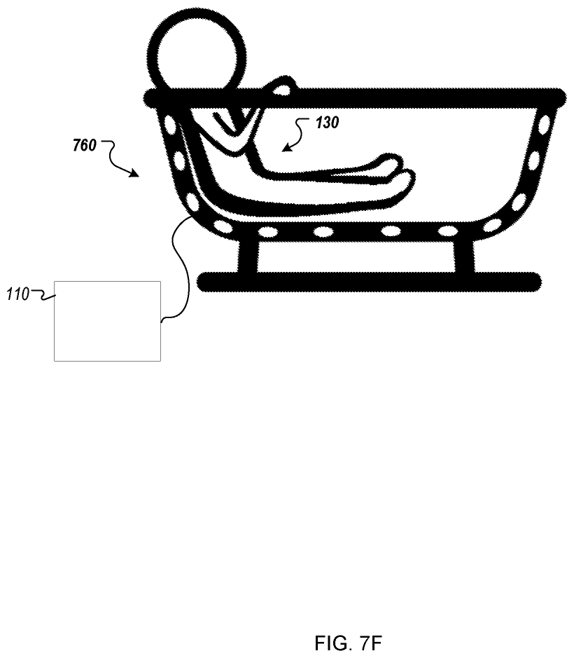

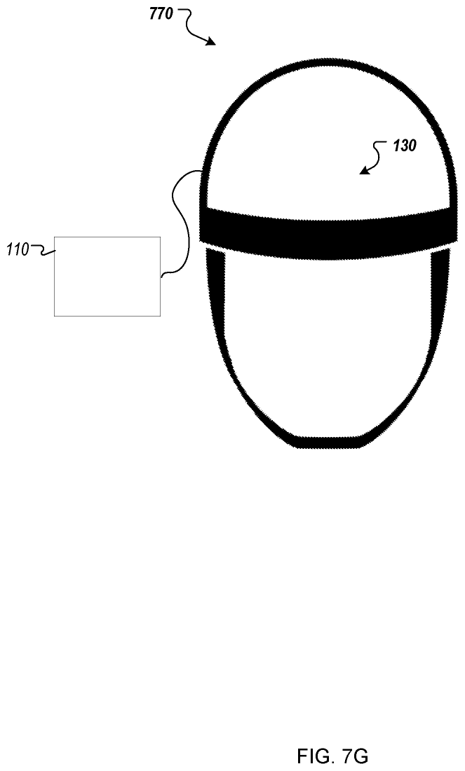

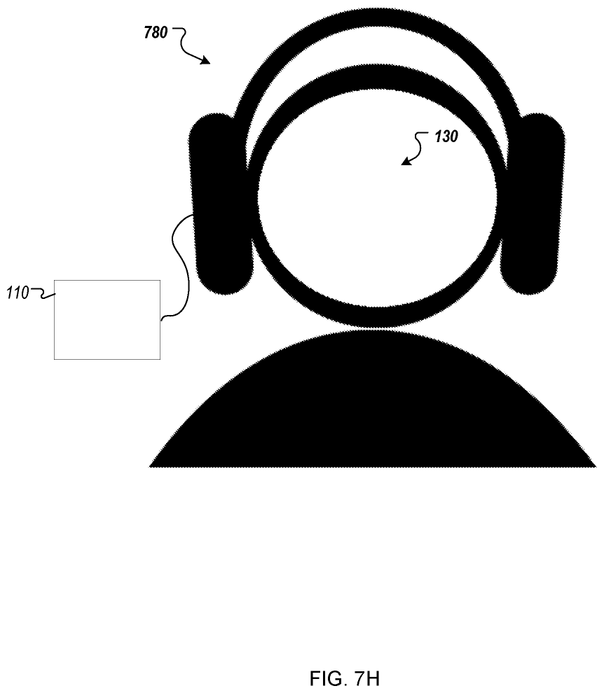

FIGS. 7A-E illustrate example form factors of a therapeutic ultrasound system that delivers an ultrasound wave to a target within a patient's body. Other form factors for the therapeutic ultrasound system described in the present application are contemplated. Devices 710, 720, 730, 740, and 750 each include arrays 106 and/or transducer array 210, and can be an implementation of systems 100 and/or 200 as described above with respect to FIGS. 1-3B. Devices 710, 720, 730, 740, and 750 can include all capabilities of the devices described in FIGS. 4A-C and 7B-F in a different form factor.

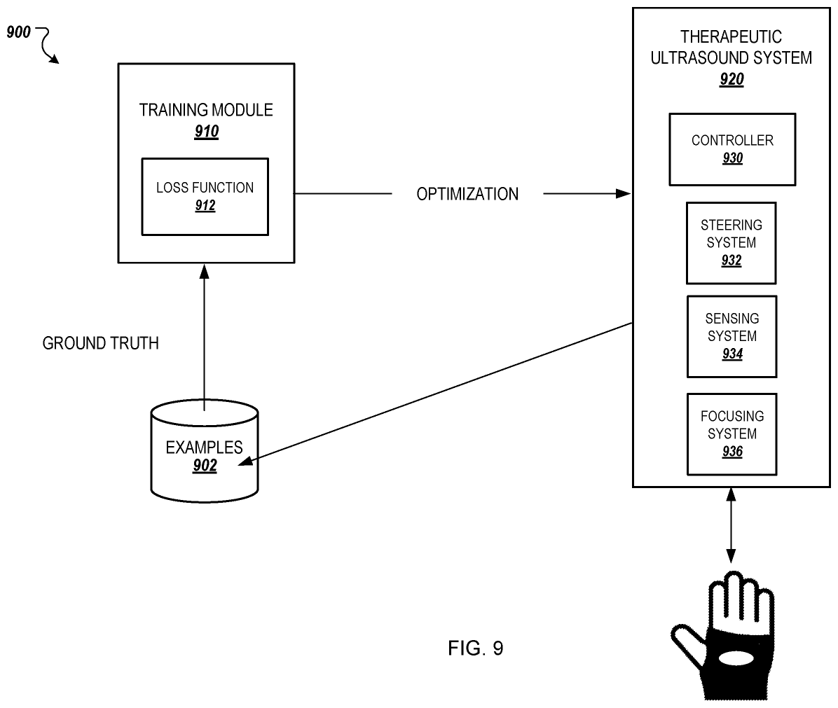

The devices illustrated in FIGS. 7A-E can be administered by a healthcare provider to a patient. In some implementations, the devices illustrated in FIGS. 7A-E can be operated by patient 120 without the supervision of a healthcare provider. For example, devices 710, 720, 730, 740, and 750 can be implemented such that arrays 106 and/or transducer array 210 can hit a wide range of positions, sizes, and shapes of targets 130 within a patient 120's body and can be made to fit many different patients. For example, devices 710, 720, 730, 740, and 750 can be provided to patients and can be adjustable by the patient, and in some implementations, can automatically calibrate to the patient and a particular target spot. Details of automatic calibration are provided below with respect to FIG. 9.

In some implementations, the therapeutic ultrasound system 100 and/or 200 can be worn while a patient is performing an activity. For example, the therapeutic ultrasound system can be incorporated into supportive equipment such as athletic braces as illustrated in FIGS. 7A-E. In some implementations, the therapeutic ultrasound system can be worn while a patient is exercising or using a particular limb or body part in which the spot 130 to be targeted is located. For example, a knee brace 7B in which the therapeutic ultrasound system 100 and/or 200 can be integrated can be worn by a patient 120 while playing soccer.

While controller 110 is depicted as separate from the devices 400, 430, 460, 710, 720, 730, 740, 750, and 760, controller 110 and associated power systems can be integrated with the devices of FIGS. 4A-C and 7A-F to provide a comfortable, more compact form factor.

In general, systems 100 and/or 200 can be implemented as a single package within a particular form factor that is best suited to a patient 120's therapeutic needs. In some implementations, systems 100 and/or 200 can be implemented as two or more devices that communicate with each other to coordinate the steering and focusing of ultrasound beams. For example, systems 100 and/or 200 can be implemented as a separate component systems: steering system 932, sensing system 934, and focusing system 936. The component systems of therapeutic ultrasound system 920 can all communicably connected and coordinate to direct ultrasound beams to a target 130 within a patient 120.

FIG. 7A illustrates a device 710 that can be worn by a patient 120 around the wrist. In this particular implementation, device 710 is in a flexible form factor that wraps around a patient's wrist and has the automatic steering and focusing systems as described in FIGS. 1-3B and 4A-C. For example, device 710 can be a wrist brace that includes a conductive layer coupling the patient's wrist with arrays 106 and/or transducer array 210. Device 710 can provide mechanical support to the patient's wrist in addition to ultrasound pulses to a target spot 130, for example, through arrays 106 and/or transducer array 210. Device 710 can be used in situations when a patient has carpal tunnel or other sources of wrist pain. For example, device 710 can be used to provide ultrasound pulses to a target nerve 130 that has been pinched and causes numbness or pain to a patient 120.

FIG. 7B illustrates example device 720 that can be worn by a patient 120 around the knee. In this particular implementation, device 720 is in a flexible form factor that wraps around a patient's knee and has the automatic steering and focusing systems as described in FIGS. 1-3B and 4A-C. Device 720 can be, for example, a knee brace that includes a conductive layer coupling the patient's knee and leg area with arrays 106 and/or transducer array 210. In this particular implementation, device 720 is shown to cover only a portion of patient 120's knee and leaves a portion of patient 120's knee exposed. In some implementations, device 720 covers the entirety of patient 120's knee. Device 720 can provide mechanical support to the patient's knee in addition to ultrasound pulses to a target spot 130, for example, through arrays 106 and/or transducer array 210. Device 720 can be used, for example, in situations when a patient has pain upstream of the knee or knee pain, such as post-operative knee pain.

FIG. 7C illustrates example device 730 that can be worn by a patient 120 around their shoulder, upper back area, or neck. In this particular implementation, device 730 is in a flexible form factor that wraps around a patient's knee and has the automatic steering and focusing systems as described in FIGS. 1-3B and 4A-C. Device 730 can be, for example, a shoulder brace that includes a conductive layer coupling the patient's shoulder or upper back with arrays 106 and/or transducer array 210. Device 730 can be worn to apply ultrasound pulses to patient 120's shoulder or upper back to address pain. For example, if a patient 120 is experiencing shoulder pain due to overuse, device 730 can be worn to apply ultrasound pulses to a target 130 within patient 120's shoulder or back.

FIG. 7D illustrates example device 740 that can be worn by a patient 120 around their back. In this particular implementation, device 740 is in a flexible form factor that wraps around a patient's back and has the automatic steering and focusing systems as described in FIGS. 1-3B and 4A-C. Device 740 can be, for example, a back brace that includes a conductive layer coupling the patient's back with arrays 106 and/or transducer array 210. Device 740 can be worn to apply ultrasound pulses to patient 120's back. For example, if a patient 120 has thrown out their back or experiences chronic back pain, device 740 can be worn to apply ultrasound pulses to a target 130 within patient 120's back.

FIG. 7E illustrates example device 750 that can be worn by a patient 120 around their ankle. In this particular implementation, device 750 is in a flexible form factor that wraps around a patient's ankle and has the automatic steering and focusing systems as described in FIGS. 1-3B and 4A-C. Device 750 can be, for example, an ankle brace that includes a conductive layer coupling the patient's back with arrays 106 and/or transducer array 210. Device 750 can be worn to apply ultrasound pulses to patient 120's ankle or leg. For example, if a patient 120 has sprained their ankle or pulled a muscle in their leg, device 750 can be worn to apply ultrasound pulses to a target 130 within patient 120's ankle.

FIG. 7F illustrates example device 760 that can be implemented as a bath in which a patient 120 is submerged. In this particular implementation, device 760 is implemented as an appliance filled with a conductive substance in which a patient 120 can be submerged. For example, device 760 can be a bath appliance that is used in settings such as spas or physical therapy locations and has the automatic steering and focusing systems as described in FIGS. 1-3B and 4A-C. Device 760 can be, for example, a tub filled with a conductive substance through which ultrasound pulses from arrays 106 and/or transducer array 210 can be directed to portions of a patient 120's body that are submerged. Device 760 can be used to apply ultrasound pulses to any portion of a patient 120's body that is submerged within the conductive substance, and can target multiple targets on or within the patient 120's body. For example, if a patient 120 is an athlete with multiple aches from practice, patient 120 can use device 760 during physical therapy to aid, or in some implementations, accelerate recovery and reduce or eliminate pain from conditions such as nerve impingements, tissue damage, and muscle knots.

Device 760 includes, for example, one or more arrays 106 and or transducer arrays 210 such that device 760 can perform comprehensive imaging and/or treatment of various portions of a patient 120's body. In some implementations, device 760 allows for 360 degree imaging of a portion of patient 120's calf in which patient 120 has chronic pain.

FIG. 7G illustrates example device 770 that can be worn by a patient 120 around their head. In this particular implementation, device 770 is in a form factor that can be worn around a patient's head and has the automatic steering and focusing systems as described in FIGS. 1-3B and 4A-C. Device 770 can be, for example, a helmet or a skull cap that fits closely against a patient's head and includes a conductive layer coupling the patient's head with arrays 106 and/or transducer array 210. Device 770 can be worn to apply ultrasound pulses to patient 120's head.

FIG. 7H illustrates example device 780 that can be worn by a patient 120 around their head. In this particular implementation, device 780 is in a flexible form factor that can be worn around a patient's head and has the automatic steering and focusing systems as described in FIGS. 1-3B and 4A-C. Device 780 can be, for example, a pair of headphones or a headband or flexible hat that fits closely against a patient's head and includes a conductive layer coupling the patient's head with arrays 106 and/or transducer array 210. Device 780 can be worn to apply ultrasound pulses to patient 120's head.

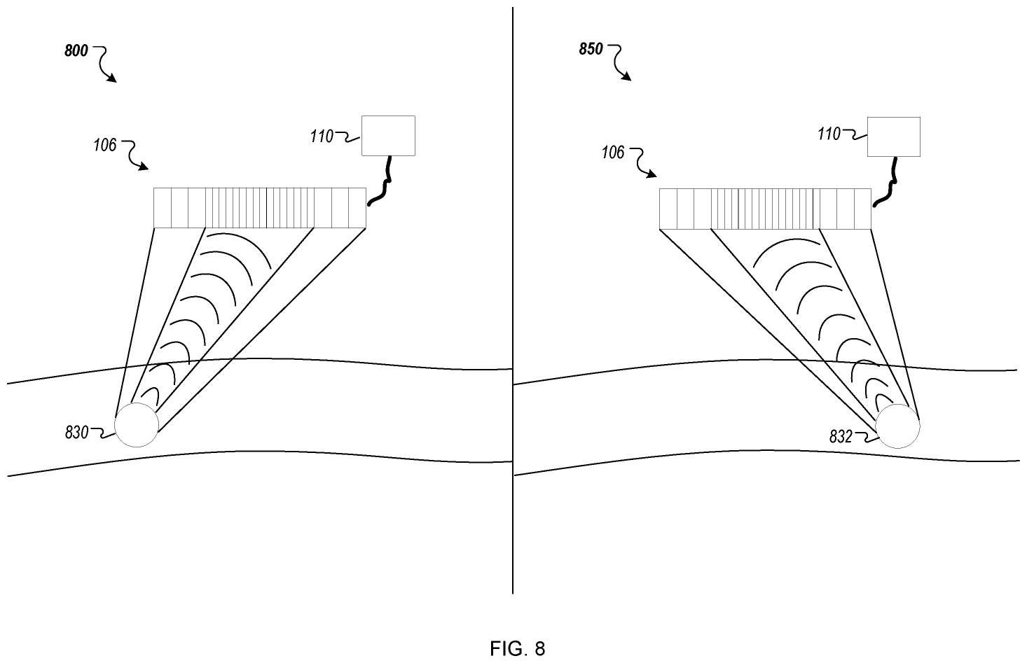

FIG. 8 is a diagram of an example targeting process for therapeutic ultrasound systems, such as systems 100 and/or 200 as described in FIGS. 1-3B.

FIG. 8 is a diagram of an example focusing process in which the therapeutic beam (e.g., from systems 100 and/or 200) focuses on different targets. In step 800, the therapeutic beam from systems 100 and/or 200 is focused on a particular therapeutic target 830. In step 850, the therapeutic beam from systems 100 and/or 200 is focused on a second, different therapeutic target 832. Step 850 can be performed a predetermined amount of time after step 800.

The process as depicted in FIG. 8 can, for example, produce an effect on a patient 120's perception of the impact of the therapeutic beam. In some implementations, moving the focus of a therapeutic beam can disrupt a pain signal travelling through a portion of a patient 120's body. Pain signals are not continuous and can be trains of impulses or temporary signals. In some implementations, moving the focus of a therapeutic beam can stop or interrupt a train of pain impulses, providing a patient 120 with relief.

The process as depicted in FIG. 8 can be implemented by controller 110 of the therapeutic ultrasound system generating control signals to direct one or more ultrasound impulses to targets 830 and 832. Nervous signals can propagate at speeds between 20 m/s to 50 m/s, and controller 110 can account for the particular propagation parameters of a particular signal, target 130, and/or patient 130.

As described above with respect to the FIG. 2, systems 100 and 200 can provide multi-pulse superposition to stack two or more ultrasound pulses such that single-pulse power thresholds are met while building up displacement with pressure or shear waves from multiple pulses with different focal locations for the safety of patient 120.

FIG. 9 is a diagram of an example block diagram of a system 900 for training a controller that steers and focuses a therapeutic ultrasound system 920. For example, system 200 can be used to train systems 100 and 200 as described with respect to FIGS. 1-2, 3A-3B, and 7A-8.

As described above, systems 100 and 200 include a controller 110 that detects the position and/or motion of one or more targets 130 within a patient 120 and controls arrays 106 and/or transducer array 210 to direct an ultrasound beam of a particular phase and intensity to the one or more targets 130. For example, controller 110 detects the position or a target 130 using, for example, ultrasound imaging and determines stimulation parameters for arrays 106 and/or transducer array 210. Steering and focusing parameter determination includes predicting a future motion of a particular pain signal or target 130 based on the detected position and/or motion of the one or more targets 130.

Examples 902 are provided to training module 910 as input to train a motion prediction model. Examples 902 can be positive examples (i.e., examples of correctly determined future motions) or negative examples (i.e., examples of incorrectly determined future motions).

Examples 902 include the ground truth future motions, or a future motion defined, or confirmed, as the correct future motion. Examples 902 include sensor information such as baseline movement patterns for a particular patient 120 and/or target 130. For example, examples 902 can include movement data for a target nerve 130 generated through activity detection performed by an imaging system and/or sensing system that communicates with system 100 and/or 200.

The ground truth indicates the actual, correct future motion indicated by the position and/or motion data. For example, a ground truth future motion can be generated and provided to training module 910 as an example 902 by detecting a position and/or motion of a target 130, determining a set of steering and focusing parameters to direct an ultrasound beam to a target 130 and/or produce a particular motion of target 130, and confirming that the resulting position and/or motion is correct. In some implementations, a human can manually verify the future motion. The future motion can be automatically detected and labelled by pulling data from a data storage medium that contains verified future motions.

The ground truth future motion can be correlated with particular inputs of examples 902 such that the inputs are labelled with the ground truth future motion. With ground truth labels, training module 910 can use examples 902 and the labels to verify model outputs of a position and/or motion predictor and continue to train the predictor to improve forward modelling of movement activity through the use of detection data from sensors 114 to predict movements in response to support configurations.

The sensor information guides the training module 910 to train the classifier to predict future movements. The training module 910 can associate patterns of movements of patient 120's body or target 130 with a future movement to map out movement ranges and activities. Inverse modelling of movement activity can be conducted by using measured responses to approximate support configurations that could produce the measured responses.

Training module 910 trains a motion prediction model to perform motion prediction. For example, training module 110 can train controller 110 to recognize target movement activity based on inputs from sensors or imaging arrays. Training module 910 refines controller 110's motion prediction model using movement data collected for a particular patient 120 or target 130.

Training module 910 trains controller 110 using a motion prediction objective function 912. Training module 910 uses motion prediction objective function 912 to train controller to predict a future motion. Objective function 912 can account for variables such as a predicted location, a predicted amplitude, a predicted frequency, and/or a predicted phase of a resulting ultrasound beam and/or motion of a target 130.

Training module 910 can train controller 110 manually or the process could be automated. For example, if an existing representation of a particular area of patient 120's musculoskeletal structure is available, the system can receive imaging data indicating movement activity of a target nerve 130 inside of patient 120 in response to an ultrasound beam directed and generated by a known set of steering and focusing parameters to identify the ground truth stimulation or excitation reaction of target nerve 130. A human can also manually verify the steering and focusing parameters.

In some implementations, controller 110 can use ultrasound location data to progressively build a detailed image of a target 130. For example, controller 110 can receive and store ultrasound data that can be combined over time to create an image of a target nerve 130 and illustrate changes in the state of the target nerve 130 due to the use of therapeutic ultrasound beams. In some implementations, controller 110 can combine sensor data to build a more detailed image of a target area 130 of patient 120. For example, controller 110 can correlate ultrasound data with bio-parameters of a patient 120, such as blood pressure and heart rate data, among other parameters.

Training module 910 uses the objective function 912 and examples 902 labelled with the ground truth future motions to train controller 110 to learn what is important for the model. Training module 910 allows controller 110 to learn by changing the weights applied to different variables to emphasize or deemphasize the importance of the variable within the model. By changing the weights applied to variables within the model, training module 910 allows the model to learn which types of information (e.g., which sensor inputs, what locations, etc.) should be more heavily weighted to produce a more accurate motion predictor.

The examples and variables can be weighted based on, for example, feedback from controller 110. For example, if controller 110 collects movement data that indicates that an ultrasound beam directed and generated by a particular set of steering and focusing parameters produces an optimal response according to objective function 912 from a target 130, then controller 110 can weigh the applied configuration more heavily than other configurations that do not achieve the objective function.

Training module 910 uses machine learning techniques to train controller 110, and can include, for example, a neural network that utilizes objective function 212 to produce parameters used in the motion prediction model. These parameters can be prediction parameters that define particular values of a model used by controller 110.

In some implementations, controller 110 uses machine learning techniques to perform imaging operations. For example, controller 110 can use a neural network to solve an image analysis problem to detect the position of a target nerve 130 within patient 120's body. Controller 110 can also use other techniques, including echolocation, to detect the location of a target 130. Such techniques are advantageously low-cost and require relatively low amounts of power because the level of detail required is low. In some implementations, controller 110 can use an infrared camera system to image a patient 120 to determine the location of a target 130.

In some implementations, controller 110 uses an objective function to achieve the specified objective. The objective can be specified by, for example, a patient 120 or a professional healthcare provider.

Controller 110's model can use active feedback data collected in response to past steering and focusing parameters to adjust future parameters. For example, controller 110 can detect a neuron signal in response to stimulation from the ultrasound beams in order to detect an incoming influx of pain signals and can modulate steering and focusing parameters to counter the incoming influx.

In some implementations, controller 110 can measure real-time target 130 response activity. For example, controller 110 can measure real-time nerve 130 displacement and optimize steering and focusing parameters based on the detected response activity. In some implementations, controller 110 can also measure bio-parameters of patient 120, including heart rate, blood pressure, and muscular activity, among other parameters, and optimize steering and focusing parameters based on the measured response activity.

As controller 110 collects excitation and target movement data, controller 110 can anonymize the data and provide the data to a central database that stores and analyzes the collected data to improve general steering and focusing models and allow controller 110 to provide more individualized strategies for each patient 120.

Controller 110 can, for example, utilize a general profile for a patient having a particular age, height, etc. Controller 110 can generalize support configurations across users who are predicted to have similar support needs. In some implementations, system 110 accepts input, from a patient or healthcare provider, of profile information such as the patient's age, height, weight, etc.

Controller 110 can utilize, for example, a “shoe size” model that is individualized to a certain extent. For example, controller 110 can use general profiles for taller people, for people who have scoliosis, etc. System 110 can alter, for example, profiles based on a patient's characteristics, such as predicting less movement of a target nerve in a shorter person.

Each model can be individualized. For example, each model can be created from a generic model by altering model parameters based on the characteristics for each patient determined from the collected data. Each model can vary for a particular patient over long periods of time and short periods of time. For example, controller 110 can track a patient's responses and adjust the steering and focusing parameters accordingly. In some implementations, each model can also be created from a model that has been individualized using a general profile and further altered for each patient. For example, a model can be created by altering model parameters based on the characteristics for each patient determined from the collected data.

Controller 110 is a closed loop system that receives response information and dynamically updates target response models based on this response information. The target response models are used to determine, for example, a target spot 130's response to particular steering and focusing parameters.

The models are trained to prioritize safety through prophylactic and reactive measures. For example, the models can operate to prevent situations in which injury can occur. The models can use aggressive predictions of a target's response to ultrasound beams, for example, to decrease a threshold amount of change in target movement data to prevent aggressive excitation that could injure a patient.

In some implementations, controller 110 uses machine learning algorithms to detect changes in parameters such as heat generated by an ultrasound beam. For example, controller 110 can use machine learning techniques to detect overheating of a particular target area 130 within a patient 120 and can send an alert to a patient 120's healthcare provider or the patient 120 to warn them.

The models can operate to detect early signs and mitigate the effects of injurious activity. For example, the models can analyze patient usage data and target response data to detect developing reliance on the systems 100 and/or 200. The models can then determine steering and focusing parameters to reduce response characteristics that indicate reliance. Controller 110 can send an alert to a patient 120's healthcare provider or the patient 120 to warn them, in case the patient 120's attention is not fully on the treatment.

In some implementations, controller 110 can limit use of systems 100 and/or 200 to particular patients such that only a specific patient 120 can use a particular system. For example, controller 110 can receive and authentic biometric data tied to a particular patient 120. In some implementations, controller 110 can, for example, use machine learning techniques to confirm the identity of a particular patient 120 based on ultrasound data collected.

Controller 110 can enforce, for example, a maximum stimulation power threshold for patient safety. In some implementations, because therapeutic ultrasound provides pain therapy, this reduction or removal of pain signals can diminish a patient's capacity to determine when an appropriate limit has been reached. Controller 110 can, for example, continuously monitor and reduce the intensity of stimulation over time or enforce time limits, among other measures. In some implementations, the safety measures can be associated with a particular activity or location. For example, if a patient 120 is detected to be on his couch and unlikely to engage in strenuous activity that could injure him, controller 110 can increase intensity limits, whereas if patient 120 is detected to be hiking in rough terrain, controller can decrease ultrasound beam intensity limits such that the patient 120 can experience a threshold level of pain as a sensory tool.

In some implementations, controller 110 can automatically shut off the systems 100 and/or 200 for the safety of patient 120. For example, controller 110 can automatically shut off systems 100 and/or 200 when a patient 120 removes the systems. In another example, if controller 110 detects that a patient 120 in a knee brace 720 is resting, controller 110 may reduce stimulation intensity of the ultrasound beams or stop beamforming entirely and increase stimulation intensity of the ultrasound beams or begin generating ultrasound beams when the patient 120 is determined to be moving. Controller 110 can, for example, receive sensor data to determine whether a patient 120 is active. For example, systems 100 and/or 200 can be integrated with form factors that include motion data sensors, such as accelerometers.

In some implementations, controller 110 can detect sleep activity of a patient 120. For example, controller 110 can receive motion and heart rate data that indicate that a patient 120 is asleep. Controller 110 can, in some implementations, automatically turn off systems 100 and/or 200 when a patient 120 is determined to be asleep. Automatic power-off features reduce the power consumption of systems 100 and/or 200 and allow for greater portability through the use of batteries.

In some implementations, controller 110 can adjust steering and focusing parameters for different compositions of targets 130 and the surrounding areas of a patient 120's body. For example, if controller 110 detects that a target 130 is proximate to a bone, controller 110 can adjust beam patterns or parameters to compensate for the material composition and conductive properties of the bone. In some implementations, controller 110 can generate control signals that generate ultrasound beams to excite a particular bone to its resonant frequency of bone to displace a connecting nerve. In some implementations, the target 130 can be inside of a patient 120's spine. Controller 110 can automatically and dynamically adjusting steering and focusing parameters to direct ultrasound beams through, for example, the cartilage between the vertebrae of patient 120's spine.

The models can monitor and enforce parameter thresholds based on reactions within patient 120. For example, the models can detect a pressure limit at which pressure cavitation may occur and restrict the steering and focus parameters to prevent generating ultrasound beams that result in cavitation. The models can also detect a thermal limit at which damage to the nerves, tissue, muscle, etc. of the patient 120 can occur due to heat from the ultrasound pulses and restrict the steering and focus parameters to prevent generating ultrasound beams that result in high levels of heat.

In some implementations, systems 100 and 200 can perform target location and stimulation as a single operation. For example, controller 110 can control a phased array of transducers, used for imagery and beamforming, to detect a target nerve 130 and stimulate the target nerve 130, thus eliminating or reducing the need for moving parts and allowing for a simple fitting process for a patient 120.

In some implementations, controller 110 uses machine learning algorithms to improve its process for detecting and locating targets 130 within patients 120. Controller 110 also uses machine learning techniques to measure target responses. For example, controller 110 can measure the displacement of a target nerve 130 in response to an ultrasound beam directed and generated by a particular set of steering and focusing parameters.

In some implementations, systems 100 and 200 can automatically calibrate to a particular patient 120 without requirement position adjustments or specialized fittings to be performed by a healthcare provider. For example, systems 100 and 200 can automatically calibrate based on the displacement of a target 130, such as a nerve. Systems 100 and 200 can, for example, automatically calibrate to stretch a target nerve 130 a certain distance on the order of microns by increasing the intensity of an ultrasound beam until a target or threshold nerve stretch distance is achieved.

In some implementations, systems 100 and 200 use a feedback system that provides objective measures of the effects of particular steering and focusing parameters and the resulting ultrasound beam. For example, systems 100 and 200 can measure target nerve 130 displacement as a function of the steering and focusing parameters. In some implementations, systems 100 and 200 can receive feedback from controller 110 itself or patient 120. For example, patient 120 can be provided with a control mechanism that allows patient 120 to control, among other parameters, the intensity, duty cycle, shape, amplitude, and frequency of the ultrasound beams being generated. In some implementations, if systems 100 and 200 allow for patient 120 input and/or control, a power envelope within which the duty cycle or pulse shape of the ultrasound beams can be changed based on patient feedback can be enforced.

In some implementations, patient feedback and control input can be received through a user interface such as a mobile application or a standalone controller. For example, a patient 120 may input feedback data through a mobile application on her smartphone that communicates with the systems 100 and/or 200 in use.

In some implementations, systems 100 and 200 can communicate with and include multiple devices. These devices can communicate wirelessly, through wired connections, and in some examples, through a body network that uses patient 120's body as a transmission medium. For example, a controller 110 can transmit control signals to one or more components of systems 100 and 200 by transmitting ultrasound pulses through a patient 120's body itself. In some implementations, systems 100 and 200 include multiple devices, and a single device can act as a master device, transmitting control signals to the other devices in the system.

A number of implementations have been described. Nevertheless, it will be understood that various modifications may be made without departing from the spirit and scope of the disclosure. For example, various forms of the flows shown above may be used, with steps re-ordered, added, or removed.

All of the functional operations described in this specification may be implemented in digital electronic circuitry, or in computer software, firmware, or hardware, including the structures disclosed in this specification and their structural equivalents, or in combinations of one or more of them. The techniques disclosed may be implemented as one or more computer program products, i.e., one or more modules of computer program instructions encoded on a computer-readable medium for execution by, or to control the operation of, data processing apparatus. The computer readable-medium may be a machine-readable storage device, a machine-readable storage substrate, a memory device, a composition of matter affecting a machine-readable propagated signal, or a combination of one or more of them. The computer-readable medium may be a non-transitory computer-readable medium. The term “data processing apparatus” encompasses all apparatus, devices, and machines for processing data, including by way of example a programmable processor, a computer, or multiple processors or computers. The apparatus may include, in addition to hardware, code that creates an execution environment for the computer program in question, e.g., code that constitutes processor firmware, a protocol stack, a database management system, an operating system, or a combination of one or more of them. A propagated signal is an artificially generated signal, e.g., a machine-generated electrical, optical, or electromagnetic signal that is generated to encode information for transmission to suitable receiver apparatus.

A computer program (also known as a program, software, software application, script, or code) may be written in any form of programming language, including compiled or interpreted languages, and it may be deployed in any form, including as a standalone program or as a module, component, subroutine, or other unit suitable for use in a computing environment. A computer program does not necessarily correspond to a file in a file system. A program may be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program may be deployed to be executed on one computer or on multiple computers that are located at one site or distributed across multiple sites and interconnected by a communication network.

The processes and logic flows described in this specification may be performed by one or more programmable processors executing one or more computer programs to perform functions by operating on input data and generating output. The processes and logic flows may also be performed by, and apparatus may also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer may be embedded in another device, e.g., a tablet computer, a mobile telephone, a personal digital assistant (PDA), a mobile audio player, a Global Positioning System (GPS) receiver, to name just a few. Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory may be supplemented by, or incorporated in, special purpose logic circuitry.

To provide for interaction with a user, the techniques disclosed may be implemented on a computer having a display device, e.g., a CRT (cathode ray tube) or LCD (liquid crystal display) monitor, for displaying information to the user and a keyboard and a pointing device, e.g., a mouse or a trackball, by which the user may provide input to the computer. Other kinds of devices may be used to provide for interaction with a user as well; for example, feedback provided to the user may be any form of sensory feedback, e.g., visual feedback, auditory feedback, or tactile feedback; and input from the user may be received in any form, including acoustic, speech, or tactile input.

Implementations may include a computing system that includes a back end component, e.g., as a data server, or that includes a middleware component, e.g., an application server, or that includes a front end component, e.g., a client computer having a graphical user interface or a Web browser through which a user may interact with an implementation of the techniques disclosed, or any combination of one or more such back end, middleware, or front end components. The components of the system may be interconnected by any form or medium of digital data communication, e.g., a communication network. Examples of communication networks include a local area network (“LAN”) and a wide area network (“WAN”), e.g., the Internet.

The computing system may include clients and servers. A client and server are generally remote from each other and typically interact through a communication network. The relationship of client and server arises by virtue of computer programs running on the respective computers and having a client-server relationship to each other.

While this specification contains many specifics, these should not be construed as limitations, but rather as descriptions of features specific to particular implementations. Certain features that are described in this specification in the context of separate implementations may also be implemented in combination in a single implementation. Conversely, various features that are described in the context of a single implementation may also be implemented in multiple implementations separately or in any suitable subcombination. Moreover, although features may be described above as acting in certain combinations and even initially claimed as such, one or more features from a claimed combination may in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems may generally be integrated together in a single software product or packaged into multiple software products.

Thus, particular implementations have been described. Other implementations are within the scope of the following claims. For example, the actions recited in the claims may be performed in a different order and still achieve desirable results.

What is claimed is:

1. A therapeutic ultrasound system, comprising:
one or more ultrasound transducer modules that are configured to generate ultrasound waves at a first power level and at a second power level, the first and second power levels being different and the second power level being sufficient to provide a therapeutic effect for a living subject; and
an electronic controller in communication with the one or more ultrasound transducer modules, the electronic controller being programmed to cause the one or more ultrasound transducer modules to generate a first ultrasound wave at the first power level and detect a reflected ultrasound wave from the subject in response to the first ultrasound wave, determine a location of a target within the subject based on the detected reflected ultrasound wave, and, after determining the location of the target, generate a second ultrasound wave at the second power level focused at the target.

2. The therapeutic ultrasound system of claim 1, wherein the one or more ultrasound transducer modules are housed in at least one of a wrist brace, a knee brace, a shoulder brace, a back brace, an ankle brace, a helmet, and a headphone.

3. The therapeutic ultrasound system of claim 1, wherein generating the second ultrasound wave at the second power level focused at the target comprises:
determining, based on the location of the target, at least one steering parameter for the one or more ultrasound transducer modules; and
dynamically adjusting, based on the at least one steering parameter, a direction of the one or more ultrasound transducer modules.

4. The therapeutic ultrasound system of claim 3, wherein generating the second ultrasound wave at the second power level focused at the target further comprises:
determining, based on the location of the target, the second power level; and
dynamically adjusting, based on the second power level, a power of the one or more ultrasound transducer modules.

5. The therapeutic ultrasound system of claim 1, wherein generating the second ultrasound wave at the second power level focused at the target comprises determining, based on the location of the target and a machine learning model, at least one steering parameter for the one or more ultrasound transducer modules.

6. The therapeutic ultrasound system of claim 1, wherein determining a location of a target within the subject based on the detected reflected ultrasound wave comprises detecting, based on the detected reflected ultrasound wave and a machine learning model, the target within the subject.

7. The therapeutic ultrasound system of claim 1, wherein the one or more ultrasound transducer modules includes an imaging array that generate ultrasound waves at the first power level, the first power level being sufficient to produce an imaging effect of the target within the subject.

8. A method for utilizing a therapeutic ultrasound system comprising:
directing, from one or more ultrasound transducer modules, a first ultrasound wave at a first power level to a subject;
detecting a reflected ultrasound wave from the subject in response to the first ultrasound wave;
determining a location of a target within the subject based on the detected reflected ultrasound wave; and
directing, using the one or more ultrasound transducer modules, a second ultrasound wave at a second power level focused at the target after determining the location of the target,
wherein the second power level is sufficient to provide a therapeutic effect for the subject.

9. The method of claim 8, wherein the one or more ultrasound transducer modules are housed in at least one of a wrist brace, a knee brace, a shoulder brace, a back brace, an ankle brace, a helmet, and a headphone.

10. The method of claim 8, further comprising:
generating the second ultrasound wave at the second power level focused at the target by determining, based on the location of the target, at least one steering parameter for the one or more ultrasound transducer modules, and
wherein directing the second ultrasound wave at a second power level focused at the target after determining the location of the target comprises dynamically adjusting, based on the at least one steering parameter, a direction of the one or more ultrasound transducer modules.

11. The method of claim 10, wherein generating the second ultrasound wave at the second power level focused at the target further comprises:
determining, based on the location of the target, the second power level; and
dynamically adjusting, based on the second power level, a power of the one or more ultrasound transducer modules.

12. The method of claim 10, wherein generating the second ultrasound wave at the second power level focused at the target comprises determining, based on the location of the target and a machine learning model, at least one steering parameter for the one or more ultrasound transducer modules.

13. The method of claim 8, wherein determining a location of a target within the subject based on the detected reflected ultrasound wave comprises detecting, based on the detected reflected ultrasound wave and a machine learning model, the target within the subject.

14. The method of claim 8, wherein the one or more ultrasound transducer modules includes an imaging array that generate ultrasound waves at the first power level, the first power level being sufficient to produce an imaging effect of the target within the subject.

15. A computer-readable storage device storing instructions that when executed by one or more processors cause the one or more processors to perform operations comprising:
directing, from one or more ultrasound transducer modules, a first ultrasound wave at a first power level to a subject;
detecting a reflected ultrasound wave from the subject in response to the first ultrasound wave;
determining a location of a target within the subject based on the detected reflected ultrasound wave; and
directing, using the one or more ultrasound transducer modules, a second ultrasound wave at a second power level focused at the target after determining the location of the target,
wherein the second power level is sufficient to provide a therapeutic effect for the subject.

16. The computer-readable storage device of claim 15, wherein the one or more ultrasound transducer modules are housed in at least one of a wrist brace, a knee brace, a shoulder brace, a back brace, an ankle brace, a helmet, and a headphone.

17. The computer-readable storage device of claim 15, the operations further comprising:
generating the second ultrasound wave at the second power level focused at the target by determining, based on the location of the target, at least one steering parameter for the one or more ultrasound transducer modules, and
wherein directing the second ultrasound wave at a second power level focused at the target after determining the location of the target comprises dynamically adjusting, based on the at least one steering parameter, a direction of the one or more ultrasound transducer modules.

18. The computer-readable storage device of claim 17, wherein generating the second ultrasound wave at the second power level focused at the target further comprises:
determining, based on the location of the target, the second power level; and
dynamically adjusting, based on the second power level, a power of the one or more ultrasound transducer modules.

19. The computer-readable storage device of claim 17, wherein generating the second ultrasound wave at the second power level focused at the target comprises determining, based on the location of the target and a machine learning model, at least one steering parameter for the one or more ultrasound transducer modules.

20. The computer-readable storage device of claim 15, wherein determining a location of a target within the subject based on the detected reflected ultrasound wave comprises detecting, based on the detected reflected ultrasound wave and a machine learning model, the target within the subject.
